(19) United States
(12) Patent Application Publication
(10) Pub. No.: US 2023/0335272 A1
(43) Pub. Date: Oct. 19, 2023

(54) BUILDING MANAGEMENT SYSTEM WITH WATER AND AIR-BASED BIOLOGICAL SENSING

(71) Applicant: Johnson Controls Tyco IP Holdings LLP, Milwaukee, WI (US)

(72) Inventor: Jonathan D. Douglas, Mequon, WI (US)

(21) Appl. No.: 18/133,831

(22) Filed: Apr. 12, 2023

Related U.S. Application Data
(60) Provisional application No. 63/330,732, filed on Apr. 13, 2022.

(51) Int. Cl. G16H 40/63; G05B 19/042

(52) U.S. Cl. CPC G16H 40/63; G05B 19/042; G05B 2219/2614

(57) ABSTRACT

A pathogen detection system for a building includes a plurality of pathogen detectors positioned in the building at a plurality of locations, the plurality of pathogen detectors configured to output pathogen data indicating whether presence of a pathogen has been detected. The pathogen detection system further includes processing circuitry configured to obtain first detection data from a first pathogen detector of the plurality of pathogen detectors positioned at a first location in the building, in response to obtaining the first detection data, analyze second detection data from a second pathogen detector of the plurality of pathogen detectors, determine a responsive action associated with an area or zone of the building based on the first detection data and the second detection data, and perform the responsive action or initiate the responsive action within the area or zone.

### CROSS-REFERENCE TO RELATED PATENT APPLICATIONS

This application claims the benefit of and priority to U.S. Provisional Application No. 63/330,732, filed Apr. 13, 2022, which is incorporated by reference herein in its entirety for all purposes.

The following applications are incorporated herein by reference in their entireties: PCT Application No. PCT/US2022/012467, filed Jan. 14, 2022; and U.S. Provisional Application No. 63/307,981, filed Feb. 8, 2022.

### BACKGROUND

The present disclosure relates generally to building management systems. More particularly, the present disclosure relates to building management systems for mitigating infection risk.

### SUMMARY

Some embodiments relate to a pathogen detection system for a building, including a plurality of pathogen detectors positioned in the building at a plurality of locations, the plurality of pathogen detectors configured to output pathogen data indicating whether presence of a pathogen has been detected, and processing circuitry configured to obtain first detection data from a first pathogen detector of the plurality of pathogen detectors positioned at a first location in the building, in response to obtaining the first detection data, analyze second detection data from a second pathogen detector of the plurality of pathogen detectors, determine a responsive action associated with an area or zone of the building based on the first detection data and the second detection data, and perform the responsive action or initiate the responsive action within the area or zone.

In some embodiments, the first pathogen detector is configured to sense the presence of the pathogen within sewage, and the second pathogen detector is configured to sense the presence of the pathogen within air.

In some embodiments, the first pathogen detector is positioned within a sewage line or sewage output of the building, and wherein the second pathogen detector is positioned on a surface of the building.

In some embodiments, a third pathogen detector is a mobile sensor configured to sense the presence of the pathogen on a surface or within air, and wherein the processing circuitry is further configured to request additional detection data from the first pathogen detector, the second pathogen detector, or the third pathogen detector based on receiving a positive detection from at least one of the plurality of pathogen detectors.

In some embodiments, the plurality of pathogen detectors includes a first sensor located in a common area or multiple areas of the building configured to detect information impacted by overall air quality or overall sewage conditions within the building, and wherein a second sensor located in the area or zone of the building configured to detect information impacted by localized air quality or localized sewage conditions within the area or zone.

In some embodiments, the pathogen detection system further includes requesting additional detection data from the mobile sensor based on receiving the second detection data from the second pathogen detector, and determining a pattern or trend in both the additional detection data and the second detection data, wherein the pattern or trend is associated with at least one of sensing the presence of the pathogen, a concentration of the pathogen, or a spread of the pathogen within the building.

In some embodiments, the processing circuitry is further configured to prioritize the responsive action based on the difference in pathogen detection between the detected information impacted by the overall air quality or the overall sewage conditions within the building and the detected information impacted by the localized air quality or the localized sewage conditions within the area or zone.

In some embodiments, the processing circuitry is further configured to monitor additional detection data from the first pathogen detector and the second pathogen detector, wherein monitoring the additional detection data includes adjusting a frequency of sampling and analysis based on at least one of previous analyses, previously collected detection data, building occupancy, or pathogen community data.

In some embodiments, the pathogen detection system is configured to determine a severity or magnitude of the pathogen in the building and/or a locality of areas of the pathogen in the building using the pathogen data.

In some embodiments, the responsive action includes a control action and the processing circuitry includes a control system configured to initiate one or more infection control sequences through operation of an infection control system of the building to perform the control action.

In some embodiments, the one or more infection control sequences including at least one of an adjustment to a fresh air intake of an air handling unit (AHU) of a heating, ventilation, or air conditioning (HVAC) system of the building, activation of one or more ultraviolet (UV) lights to disinfect return air from a zone of the building, or initiating one or more filtration techniques to filter air in the building.

Some embodiments relate to a pathogen detection system for a building, including a first pathogen detector configured to obtain a first sample, the first pathogen detector positioned at a first location in the building, a second pathogen detector configured to obtain a second sample, wherein the first pathogen detector and the second pathogen detector output pathogen data indicating whether presence of a pathogen has been detected, and a detection controller configured to assess the pathogen data and determine a responsive action associated with an area or zone of the building based on the first sample and the second sample.

In some embodiments, the first pathogen detector is a sewage sampling system configured to take a sample of sewage from a sewage line or a sewage outlet of the building and sense the presence of the pathogen within the sample of sewage.

In some embodiments, the second pathogen detector is an air sensor configured to take a sample of air from one or more areas or zones of the building and sense and sense the presence of the pathogen within the sample of air.

In some embodiments, the pathogen detection system further includes a mobile sensor configured to sense the presence of the pathogen on a surface or within air, and wherein the detection controller is further configured to request additional detection data from the first pathogen detector, the second pathogen detector, or the mobile sensor based on receiving a positive detection from at least one of the first pathogen detector, the second pathogen detector, or the mobile sensor.

In some embodiments, the first pathogen detector is located in a common area or multiple areas of the building and configured to detect information impacted by overall air quality or overall sewage conditions within the building, and the second pathogen detector is located in the area or zone of the building and configured to detect information impacted by localized air quality or localized sewage conditions within the area or zone.

In some embodiments, the detection controller is further configured to prioritize the responsive action based on the difference in pathogen detection between the detected information impacted by the overall air quality or the overall sewage conditions within the building and the detected information impacted by the localized air quality or the localized sewage conditions within the area or zone.

In some embodiments, the pathogen detection system further includes a transceiver configured to transmit a message to an external system and the external system configured to take one or more additional actions to detect or limit a spread of the pathogen in the building responsive to receiving the message from the transceiver.

In some embodiments, the detection controller is further configured to generate a message indicating at least one of a presence or absence of the pathogen in the first sample or the second sample, and update a plurality of locations of the first pathogen detector or the second pathogen detector within the building based on the presence of the pathogen in the first sample or the second sample.

In some embodiments, the detection controller is further configured to perform the responsive action or initiate the responsive action within the area or zone.

Some embodiments relate to a method including obtaining, by a pathogen detection system, first detection data from a first pathogen detector of a plurality of pathogen detectors positioned at a first location in a building, in response to obtaining the first detection data, analyzing, by the pathogen detection system, second detection data from a second pathogen detector of the plurality of pathogen detectors, determining, by the pathogen detection system, a responsive action associated with an area or zone of the building based on the first detection data and the second detection data, and performing, by the pathogen detection system, the responsive action or initiate the responsive action within the area or zone.

### BRIEF DESCRIPTION OF THE DRAWINGS

FIG. 1 is a drawing of a building equipped with a HVAC system, according to some embodiments.

FIG. 2 is a block diagram of a waterside system which can be used to serve the building of FIG. 1, according to some embodiments.

FIG. 3 is a block diagram of an airside system which can be used to serve the building of FIG. 1, according to some embodiments.

FIG. 4 is a block diagram of a building management system (BMS) which can be used to monitor and control the building of FIG. 1, according to some embodiments.

FIG. 5 is a block diagram of a pathogen detection system for a building, according to some embodiments.

FIG. 6 is a block diagram of a detection controller of the pathogen detection system of FIG. 5, according to some embodiments.

FIG. 7 is a flow diagram of a process for performing pathogen detection and responsive actions, according to some embodiments.

FIG. 8 is a flow diagram of a process for performing pathogen detection and responsive actions, according to some embodiments.

It will be recognized that some or all of the figures are schematic representations for purposes of illustration. The figures are provided for the purpose of illustrating one or more embodiments with the explicit understanding that they will not be used to limit the scope or the meaning of the claims.

### DETAILED DESCRIPTION

### Overview

Referring generally to the FIGURES, systems and methods for pathogen detection for a building are shown. The pathogen detection system can include pathogen detectors positioned at one or more locations (e.g., throughout) in or near the building that are configured to sense a presence and/or a type of pathogen in the building. A detection controller can obtain detection results from each of the pathogen detectors. The detection controller can, in some implementations, determine a magnitude or severity of pathogenic outbreak in the building. Additionally, the detection controller can, in various implementations, detect presence of pathogens and/or locations of the detection. In some implementations, the detection controller can determine one or more responsive actions based on any of, or any combination of, pathogen detection, locations of pathogen detection in the building, a type of pathogen detected in the building, a number of instances of pathogen detection, the magnitude or severity of the pathogenic outbreak in the building, etc. The detection controller can communicate with a variety of systems of the building, systems associated with the building, sub-systems, etc., to implement the responsive actions. The responsive actions may differ based on application of the systems and methods (e.g., different types of buildings or facilities in which the systems and methods are implemented). The responsive actions can be applied across the entire building, or may be targeted to specific zones, areas, or locations in the building (based on the locations of pathogen detection in the building).

In some implementations of the present disclosure, multiple pathogen detectors may be used in combination to enhance detection of and/or response to pathogens. In some such implementations, one or more airborne pathogen detectors may be used to sense the presence of one or more types of pathogens in the air, and one or more sewage pathogen detectors may be used to sense the presence of one or more pathogens in sewage. It should be understood that these are merely two examples of different types of pathogen detectors, and the present disclosure contemplates the use of any of a variety of different types of pathogen detectors in combination (e.g., airborne detectors, sewage detectors, water detectors configured to sense the presence of pathogens in water, surface detectors configured to sense the presence of pathogens on surfaces, patient sampling detectors configured to sense the presence of pathogens from samples taken from occupants/patients, etc.).

For example, in some implementations, an airborne pathogen detector may be used to detect the presence of a pathogen in sewage (e.g., by placing the detector in or near the sewage, i.e., sensing the pathogen in the sewage or in the air proximate to the sewage). In some implementations, a sewage pathogen detector could be used to sense the presence of a pathogen in the sewage, and in response to detecting the presence of the pathogen, data from one or more airborne pathogen detectors could be obtained and/or analyzed to determine locations and/or severity of spread of the pathogen in a building. In some implementations, the airborne pathogen detectors could be activated responsive to detection of the presence of the pathogen by the sewage pathogen detectors. In some implementations, the sewage pathogen detectors and/or airborne pathogen detectors could obtain samples to be processed off-premises, and in some implementations, the sewage pathogen detectors and/or airborne pathogen detectors could process samples on-premises (e.g., at the location of the detectors or at a separate location within the building).

While portions of the present disclosure discuss airborne and sewage-based pathogen detectors, it should be understood that the use of any of the above-identified pathogen detectors or any other detectors is contemplated by, and falls within the scope of, the present disclosure. Additionally, it should be understood that features of the present disclosure could be used to detect any type of substance, and not just pathogens, in various example implementations. For example, in some implementations, features of the present disclosure may use one or more types of detectors to detect the presence of toxins, VOCs, particulates (e.g., airborne particulates), or any other type of substance, and all such implementations are contemplated by, and falls within the scope of, the present disclosure.

### Building HVAC Systems and Building Management Systems

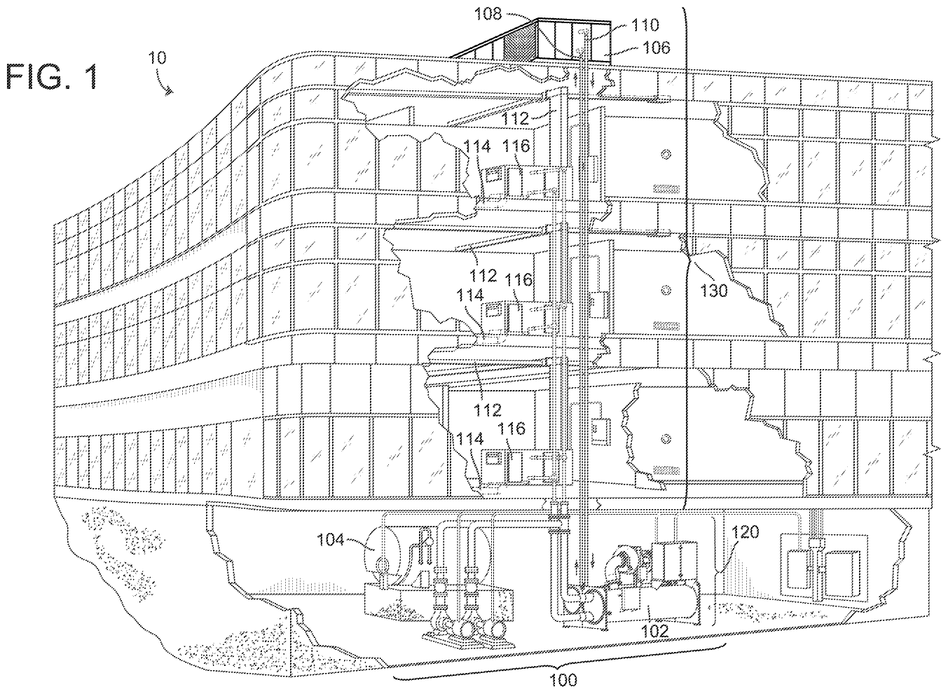

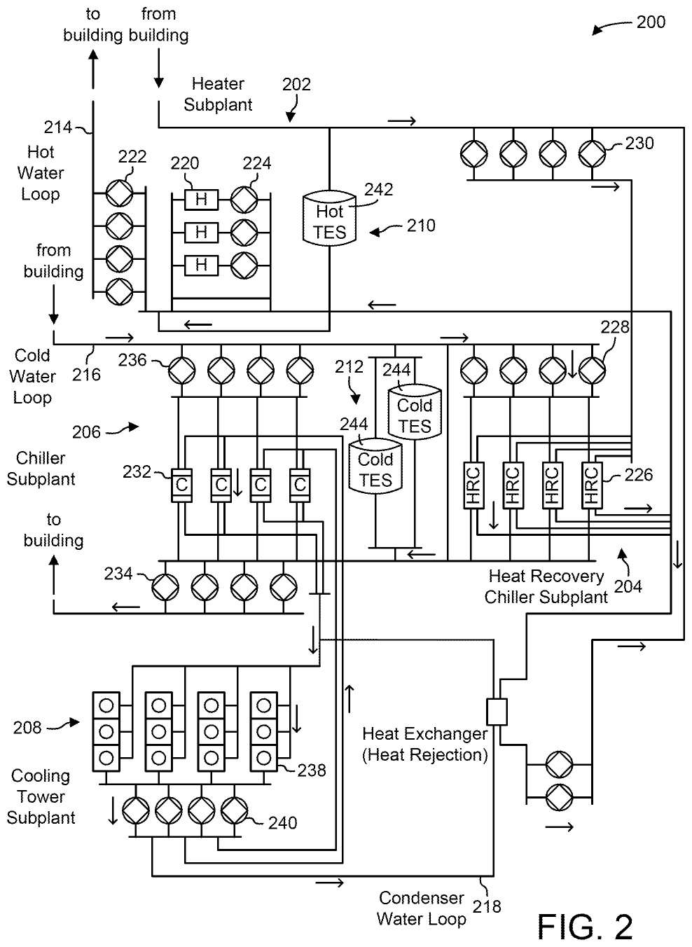

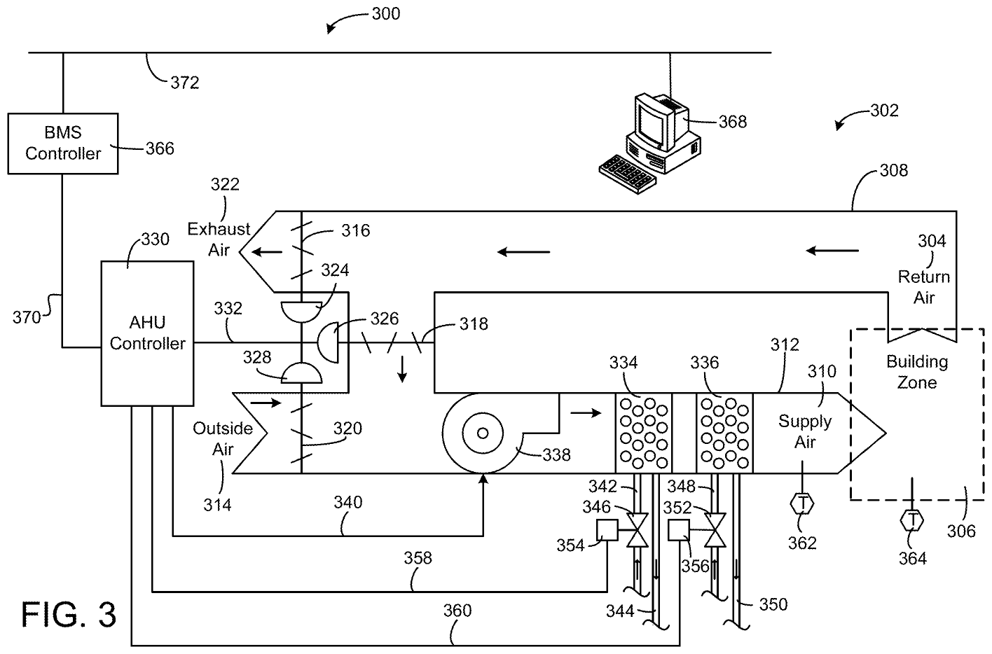

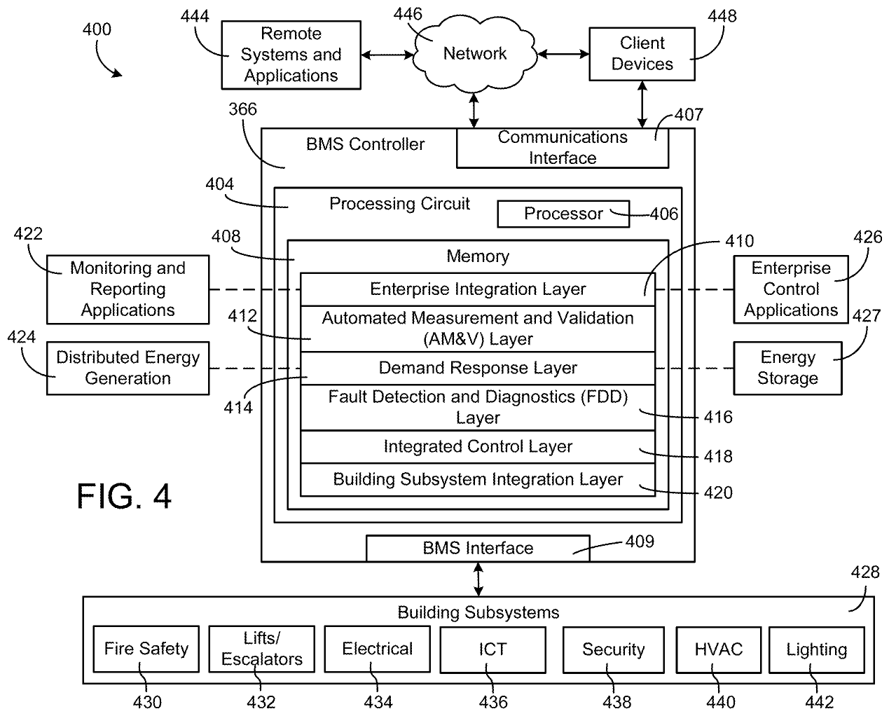

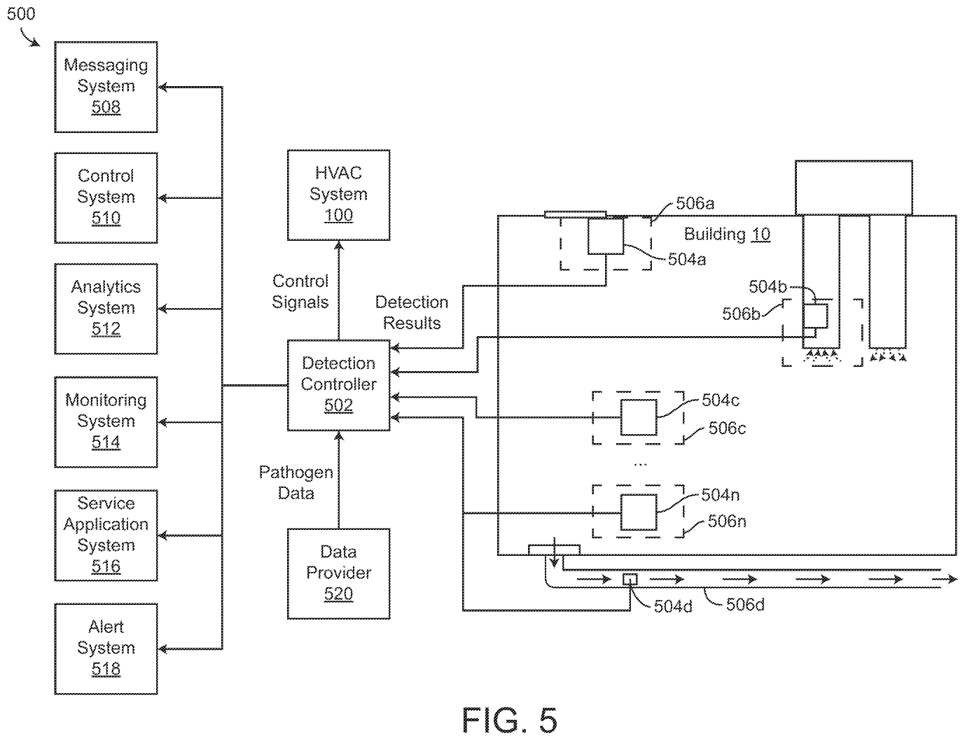

Referring now to FIGS. 1-5, several building management systems (BMS) and HVAC systems in which the systems and methods of the present disclosure can be implemented are shown, according to some embodiments. In brief overview, FIG. 1 shows a building 10 equipped with a HVAC system 100. FIG. 2 is a block diagram of a waterside system 200 which can be used to serve building 10. FIG. 3 is a block diagram of an airside system 300 which can be used to serve building 10. FIG. 4 is a block diagram of a BMS which can be used to monitor and control building 10. FIG. 5 is a block diagram of another BMS which can be used to monitor and control building 10.

### Building and HVAC System

Referring particularly to FIG. 1, a perspective view of a building 10 is shown. Building 10 is served by a BMS. A BMS is, in general, a system of devices configured to control, monitor, and manage equipment in or around a building or building area. A BMS can include, for example, a HVAC system, a security system, a lighting system, a fire alerting system, any other system that is capable of managing building functions or devices, or any combination thereof.

The BMS that serves building 10 includes a HVAC system 100. HVAC system 100 can include a plurality of HVAC devices (e.g., heaters, chillers, air handling units, pumps, fans, thermal energy storage, etc.) configured to provide heating, cooling, ventilation, or other services for building 10. For example, HVAC system 100 is shown to include a waterside system 120 and an airside system 130. Waterside system 120 may provide a heated or chilled fluid to an air handling unit of airside system 130. Airside system 130 may use the heated or chilled fluid to heat or cool an airflow provided to building 10. An exemplary waterside system and airside system which can be used in HVAC system 100 are described in greater detail with reference to FIGS. 2-3.

HVAC system 100 is shown to include a chiller 102, a boiler 104, and a rooftop air handling unit (AHU) 106. Waterside system 120 may use boiler 104 and chiller 102 to heat or cool a working fluid (e.g., water, glycol, etc.) and may circulate the working fluid to AHU 106. In various embodiments, the HVAC devices of waterside system 120 can be located in or around building 10 (as shown in FIG. 1) or at an offsite location such as a central plant (e.g., a chiller plant, a steam plant, a heat plant, etc.). The working fluid can be heated in boiler 104 or cooled in chiller 102, depending on whether heating or cooling is required in building 10. Boiler 104 may add heat to the circulated fluid, for example, by burning a combustible material (e.g., natural gas) or using an electric heating element. Chiller 102 may place the circulated fluid in a heat exchange relationship with another fluid (e.g., a refrigerant) in a heat exchanger (e.g., an evaporator) to absorb heat from the circulated fluid. The working fluid from chiller 102 and/or boiler 104 can be transported to AHU 106 via piping 108.

AHU 106 may place the working fluid in a heat exchange relationship with an airflow passing through AHU 106 (e.g., via one or more stages of cooling coils and/or heating coils). The airflow can be, for example, outside air, return air from within building 10, or a combination of both. AHU 106 may transfer heat between the airflow and the working fluid to provide heating or cooling for the airflow. For example, AHU 106 can include one or more fans or blowers configured to pass the airflow over or through a heat exchanger containing the working fluid. The working fluid may then return to chiller 102 or boiler 104 via piping 110.

Airside system 130 may deliver the airflow supplied by AHU 106 (i.e., the supply airflow) to building 10 via air supply ducts 112 and may provide return air from building 10 to AHU 106 via air return ducts 114. In some embodiments, airside system 130 includes multiple variable air volume (VAV) units 116. For example, airside system 130 is shown to include a separate VAV unit 116 on each floor or zone of building 10. VAV units 116 can include dampers or other flow control elements that can be operated to control an amount of the supply airflow provided to individual zones of building 10. In other embodiments, airside system 130 delivers the supply airflow into one or more zones of building 10 (e.g., via supply ducts 112) without using intermediate VAV units 116 or other flow control elements. AHU 106 can include various sensors (e.g., temperature sensors, pressure sensors, etc.) configured to measure attributes of the supply airflow. AHU 106 may receive input from sensors located within AHU 106 and/or within the building zone and may adjust the flow rate, temperature, or other attributes of the supply airflow through AHU 106 to achieve setpoint conditions for the building zone.

### Waterside System

Referring now to FIG. 2, a block diagram of a waterside system 200 is shown, according to some embodiments. In various embodiments, waterside system 200 may supplement or replace waterside system 120 in HVAC system 100 or can be implemented separate from HVAC system 100. When implemented in HVAC system 100, waterside system 200 can include a subset of the HVAC devices in HVAC system 100 (e.g., boiler 104, chiller 102, pumps, valves, etc.) and may operate to supply a heated or chilled fluid to AHU 106. The HVAC devices of waterside system 200 can be located within building 10 (e.g., as components of waterside system 120) or at an offsite location such as a central plant.

In FIG. 2, waterside system 200 is shown as a central plant having a plurality of subplants 202-212. Subplants 202-212 are shown to include a heater subplant 202, a heat recovery chiller subplant 204, a chiller subplant 206, a cooling tower subplant 208, a hot thermal energy storage (TES) subplant 210, and a cold thermal energy storage (TES) subplant 212. Subplants 202-212 consume resources (e.g., water, natural gas, electricity, etc.) from utilities to serve thermal energy loads (e.g., hot water, cold water, heating, cooling, etc.) of a building or campus. For example, heater subplant 202 can be configured to heat water in a hot water loop 214 that circulates the hot water between heater subplant 202 and building 10. Chiller subplant 206 can be configured to chill water in a cold water loop 216 that circulates the cold water between chiller subplant 206 building 10. Heat recovery chiller subplant 204 can be configured to transfer heat from cold water loop 216 to hot water loop 214 to provide additional heating for the hot water and additional cooling for the cold water. Condenser water loop 218 may absorb heat from the cold water in chiller subplant 206 and reject the absorbed heat in cooling tower subplant 208 or transfer the absorbed heat to hot water loop 214. Hot TES subplant 210 and cold TES subplant 212 may store hot and cold thermal energy, respectively, for subsequent use.

Hot water loop 214 and cold water loop 216 may deliver the heated and/or chilled water to air handlers located on the rooftop of building 10 (e.g., AHU 106) or to individual floors or zones of building 10 (e.g., VAV units 116). The air handlers push air past heat exchangers (e.g., heating coils or cooling coils) through which the water flows to provide heating or cooling for the air. The heated or cooled air can be delivered to individual zones of building 10 to serve thermal energy loads of building 10. The water then returns to subplants 202-212 to receive further heating or cooling.

Although subplants 202-212 are shown and described as heating and cooling water for circulation to a building, it is understood that any other type of working fluid (e.g., glycol, CO2, etc.) can be used in place of or in addition to water to serve thermal energy loads. In other embodiments, subplants 202-212 may provide heating and/or cooling directly to the building or campus without requiring an intermediate heat transfer fluid. These and other variations to waterside system 200 are within the teachings of the present disclosure.

Each of subplants 202-212 can include a variety of equipment configured to facilitate the functions of the subplant. For example, heater subplant 202 is shown to include a plurality of heating elements 220 (e.g., boilers, electric heaters, etc.) configured to add heat to the hot water in hot water loop 214. Heater subplant 202 is also shown to include several pumps 222 and 224 configured to circulate the hot water in hot water loop 214 and to control the flow rate of the hot water through individual heating elements 220. Chiller subplant 206 is shown to include a plurality of chillers 232 configured to remove heat from the cold water in cold water loop 216. Chiller subplant 206 is also shown to include several pumps 234 and 236 configured to circulate the cold water in cold water loop 216 and to control the flow rate of the cold water through individual chillers 232.

Heat recovery chiller subplant 204 is shown to include a plurality of heat recovery heat exchangers 226 (e.g., refrigeration circuits) configured to transfer heat from cold water loop 216 to hot water loop 214. Heat recovery chiller subplant 204 is also shown to include several pumps 228 and 230 configured to circulate the hot water and/or cold water through heat recovery heat exchangers 226 and to control the flow rate of the water through individual heat recovery heat exchangers 226. Cooling tower subplant 208 is shown to include a plurality of cooling towers 238 configured to remove heat from the condenser water in condenser water loop 218. Cooling tower subplant 208 is also shown to include several pumps 240 configured to circulate the condenser water in condenser water loop 218 and to control the flow rate of the condenser water through individual cooling towers 238.

Hot TES subplant 210 is shown to include a hot TES tank 242 configured to store the hot water for later use. Hot TES subplant 210 may also include one or more pumps or valves configured to control the flow rate of the hot water into or out of hot TES tank 242. Cold TES subplant 212 is shown to include cold TES tanks 244 configured to store the cold water for later use. Cold TES subplant 212 may also include one or more pumps or valves configured to control the flow rate of the cold water into or out of cold TES tanks 244.

In some embodiments, one or more of the pumps in waterside system 200 (e.g., pumps 222, 224, 228, 230, 234, 236, and/or 240) or pipelines in waterside system 200 include an isolation valve associated therewith. Isolation valves can be integrated with the pumps or positioned upstream or downstream of the pumps to control the fluid flows in waterside system 200. In various embodiments, waterside system 200 can include more, fewer, or different types of devices and/or subplants based on the particular configuration of waterside system 200 and the types of loads served by waterside system 200.

### Airside System

Referring now to FIG. 3, a block diagram of an airside system 300 is shown, according to some embodiments. In various embodiments, airside system 300 may supplement or replace airside system 130 in HVAC system 100 or can be implemented separate from HVAC system 100. When implemented in HVAC system 100, airside system 300 can include a subset of the HVAC devices in HVAC system 100 (e.g., AHU 106, VAV units 116, ducts 112-114, fans, dampers, etc.) and can be located in or around building 10. Airside system 300 may operate to heat or cool an airflow provided to building 10 using a heated or chilled fluid provided by waterside system 200.

In FIG. 3, airside system 300 is shown to include an economizer-type air handling unit (AHU) 302. Economizer-type AHUs vary the amount of outside air and return air used by the air handling unit for heating or cooling. For example, AHU 302 may receive return air 304 from building zone 306 via return air duct 308 and may deliver supply air 310 to building zone 306 via supply air duct 312. In some embodiments, AHU 302 is a rooftop unit located on the roof of building 10 (e.g., AHU 106 as shown in FIG. 1) or otherwise positioned to receive both return air 304 and outside air 314. AHU 302 can be configured to operate exhaust air damper 316, mixing damper 318, and outside air damper 320 to control an amount of outside air 314 and return air 304 that combine to form supply air 310. Any return air 304 that does not pass through mixing damper 318 can be exhausted from AHU 302 through exhaust damper 316 as exhaust air 322.

Each of dampers 316-320 can be operated by an actuator. For example, exhaust air damper 316 can be operated by actuator 324, mixing damper 318 can be operated by actuator 326, and outside air damper 320 can be operated by actuator 328. Actuators 324-328 may communicate with an AHU controller 330 via a communications link 332. Actuators 324-328 may receive control signals from AHU controller 330 and may provide feedback signals to AHU controller 330. Feedback signals can include, for example, an indication of a current actuator or damper position, an amount of torque or force exerted by the actuator, diagnostic information (e.g., results of diagnostic tests performed by actuators 324-328), status information, commissioning information, configuration settings, calibration data, and/or other types of information or data that can be collected, stored, or used by actuators 324-328. AHU controller 330 can be an economizer controller configured to use one or more control algorithms (e.g., state-based algorithms, extremum seeking control (ESC) algorithms, proportional-integral (PI) control algorithms, proportional-integral-derivative (PID) control algorithms, model predictive control (MPC) algorithms, feedback control algorithms, etc.) to control actuators 324-328.

Still referring to FIG. 3, AHU 302 is shown to include a cooling coil 334, a heating coil 336, and a fan 338 positioned within supply air duct 312. Fan 338 can be configured to force supply air 310 through cooling coil 334 and/or heating coil 336 and provide supply air 310 to building zone 306. AHU controller 330 may communicate with fan 338 via communications link 340 to control a flow rate of supply air 310. In some embodiments, AHU controller 330 controls an amount of heating or cooling applied to supply air 310 by modulating a speed of fan 338.

Cooling coil 334 may receive a chilled fluid from waterside system 200 (e.g., from cold water loop 216) via piping 342 and may return the chilled fluid to waterside system 200 via piping 344. Valve 346 can be positioned along piping 342 or piping 344 to control a flow rate of the chilled fluid through cooling coil 334. In some embodiments, cooling coil 334 includes multiple stages of cooling coils that can be independently activated and deactivated (e.g., by AHU controller 330, by BMS controller 366, etc.) to modulate an amount of cooling applied to supply air 310.

Heating coil 336 may receive a heated fluid from waterside system 200 (e.g., from hot water loop 214) via piping 348 and may return the heated fluid to waterside system 200 via piping 350. Valve 352 can be positioned along piping 348 or piping 350 to control a flow rate of the heated fluid through heating coil 336. In some embodiments, heating coil 336 includes multiple stages of heating coils that can be independently activated and deactivated (e.g., by AHU controller 330, by BMS controller 366, etc.) to modulate an amount of heating applied to supply air 310.

Each of valves 346 and 352 can be controlled by an actuator. For example, valve 346 can be controlled by actuator 354 and valve 352 can be controlled by actuator 356. Actuators 354-356 may communicate with AHU controller 330 via communications links 358-360. Actuators 354-356 may receive control signals from AHU controller 330 and may provide feedback signals to controller 330. In some embodiments, AHU controller 330 receives a measurement of the supply air temperature from a temperature sensor 362 positioned in supply air duct 312 (e.g., downstream of cooling coil 334 and/or heating coil 336). AHU controller 330 may also receive a measurement of the temperature of building zone 306 from a temperature sensor 364 located in building zone 306.

In some embodiments, AHU controller 330 operates valves 346 and 352 via actuators 354-356 to modulate an amount of heating or cooling provided to supply air 310 (e.g., to achieve a setpoint temperature for supply air 310 or to maintain the temperature of supply air 310 within a setpoint temperature range). The positions of valves 346 and 352 affect the amount of heating or cooling provided to supply air 310 by cooling coil 334 or heating coil 336 and may correlate with the amount of energy consumed to achieve a desired supply air temperature. AHU 330 may control the temperature of supply air 310 and/or building zone 306 by activating or deactivating coils 334-336, adjusting a speed of fan 338, or a combination of both.

Still referring to FIG. 3, airside system 300 is shown to include a building management system (BMS) controller 366 and a client device 368. BMS controller 366 can include one or more computer systems (e.g., servers, supervisory controllers, subsystem controllers, etc.) that serve as system level controllers, application or data servers, head nodes, or master controllers for airside system 300, waterside system 200, HVAC system 100, and/or other controllable systems that serve building 10. BMS controller 366 may communicate with multiple downstream building systems or subsystems (e.g., HVAC system 100, a security system, a lighting system, waterside system 200, etc.) via a communications link 370 according to like or disparate protocols (e.g., LON, BACnet, etc.). In various embodiments, AHU controller 330 and BMS controller 366 can be separate (as shown in FIG. 3) or integrated. In an integrated implementation, AHU controller 330 can be a software module configured for execution by a processor of BMS controller 366.

In some embodiments, AHU controller 330 receives information from BMS controller 366 (e.g., commands, setpoints, operating boundaries, etc.) and provides information to BMS controller 366 (e.g., temperature measurements, valve or actuator positions, operating statuses, diagnostics, etc.). For example, AHU controller 330 may provide BMS controller 366 with temperature measurements from temperature sensors 362-364, equipment on/off states, equipment operating capacities, and/or any other information that can be used by BMS controller 366 to monitor or control a variable state or condition within building zone 306.

Client device 368 can include one or more human-machine interfaces or client interfaces (e.g., graphical user interfaces, reporting interfaces, text-based computer interfaces, client-facing web services, web servers that provide pages to web clients, etc.) for controlling, viewing, or otherwise interacting with HVAC system 100, its subsystems, and/or devices. Client device 368 can be a computer workstation, a client terminal, a remote or local interface, or any other type of user interface device. Client device 368 can be a stationary terminal or a mobile device. For example, client device 368 can be a desktop computer, a computer server with a user interface, a laptop computer, a tablet, a smartphone, a PDA, or any other type of mobile or non-mobile device. Client device 368 may communicate with BMS controller 366 and/or AHU controller 330 via communications link 372.

### Building Management Systems

Referring now to FIG. 4, a block diagram of a building management system (BMS) 400 is shown, according to some embodiments. BMS 400 can be implemented in building 10 to automatically monitor and control various building functions. BMS 400 is shown to include BMS controller 366 and a plurality of building subsystems 428. Building subsystems 428 are shown to include a building electrical subsystem 434, an information communication technology (ICT) subsystem 436, a security subsystem 438, a HVAC subsystem 440, a lighting subsystem 442, a lift/escalators subsystem 432, and a fire safety subsystem 430. In various embodiments, building subsystems 428 can include fewer, additional, or alternative subsystems. For example, building subsystems 428 may also or alternatively include a refrigeration subsystem, an advertising or signage subsystem, a cooking subsystem, a vending subsystem, a printer or copy service subsystem, or any other type of building subsystem that uses controllable equipment and/or sensors to monitor or control building 10. In some embodiments, building subsystems 428 include waterside system 200 and/or airside system 300, as described with reference to FIGS. 2-3.

Each of building subsystems 428 can include any number of devices, controllers, and connections for completing its individual functions and control activities. HVAC subsystem 440 can include many of the same components as HVAC system 100, as described with reference to FIGS. 1-3. For example, HVAC subsystem 440 can include a chiller, a boiler, any number of air handling units, economizers, field controllers, supervisory controllers, actuators, temperature sensors, and other devices for controlling the temperature, humidity, airflow, or other variable conditions within building 10. Lighting subsystem 442 can include any number of light fixtures, ballasts, lighting sensors, dimmers, or other devices configured to controllably adjust the amount of light provided to a building space. Security subsystem 438 can include occupancy sensors, video surveillance cameras, digital video recorders, video processing servers, intrusion detection devices, access control devices and servers, or other security-related devices.

Still referring to FIG. 4, BMS controller 366 is shown to include a communications interface 407 and a BMS interface 409. Interface 407 may facilitate communications between BMS controller 366 and external applications (e.g., monitoring and reporting applications 422, enterprise control applications 426, remote systems and applications 444, applications residing on client devices 448, etc.) for allowing user control, monitoring, and adjustment to BMS controller 366 and/or subsystems 428. Interface 407 may also facilitate communications between BMS controller 366 and client devices 448. BMS interface 409 may facilitate communications between BMS controller 366 and building subsystems 428 (e.g., HVAC, lighting security, lifts, power distribution, business, etc.).

Interfaces 407, 409 can be or include wired or wireless communications interfaces (e.g., jacks, antennas, transmitters, receivers, transceivers, wire terminals, etc.) for conducting data communications with building subsystems 428 or other external systems or devices. In various embodiments, communications via interfaces 407, 409 can be direct (e.g., local wired or wireless communications) or via a communications network 446 (e.g., a WAN, the Internet, a cellular network, etc.). For example, interfaces 407, 409 can include an Ethernet card and port for sending and receiving data via an Ethernet-based communications link or network. In another example, interfaces 407, 409 can include a Wi-Fi transceiver for communicating via a wireless communications network. In another example, one or both of interfaces 407, 409 can include cellular or mobile phone communications transceivers. In one embodiment, communications interface 407 is a power line communications interface and BMS interface 409 is an Ethernet interface. In other embodiments, both communications interface 407 and BMS interface 409 are Ethernet interfaces or are the same Ethernet interface.

Still referring to FIG. 4, BMS controller 366 is shown to include a processing circuit 404 including a processor 406 and memory 408. Processing circuit 404 can be communicably connected to BMS interface 409 and/or communications interface 407 such that processing circuit 404 and the various components thereof can send and receive data via interfaces 407, 409. Processor 406 can be implemented as a general purpose processor, an application specific integrated circuit (ASIC), one or more field programmable gate arrays (FPGAs), a group of processing components, or other suitable electronic processing components.

Memory 408 (e.g., memory, memory unit, storage device, etc.) can include one or more devices (e.g., RAM, ROM, Flash memory, hard disk storage, etc.) for storing data and/or computer code for completing or facilitating the various processes, layers and modules described in the present application. Memory 408 can be or include volatile memory or non-volatile memory. Memory 408 can include database components, object code components, script components, or any other type of information structure for supporting the various activities and information structures described in the present application. According to some embodiments, memory 408 is communicably connected to processor 406 via processing circuit 404 and includes computer code for executing (e.g., by processing circuit 404 and/or processor 406) one or more processes described herein.

In some embodiments, BMS controller 366 is implemented within a single computer (e.g., one server, one housing, etc.). In various other embodiments BMS controller 366 can be distributed across multiple servers or computers (e.g., that can exist in distributed locations). Further, while FIG. 4 shows applications 422 and 426 as existing outside of BMS controller 366, in some embodiments, applications 422 and 426 can be hosted within BMS controller 366 (e.g., within memory 408).

Still referring to FIG. 4, memory 408 is shown to include an enterprise integration layer 410, an automated measurement and validation (AM&V) layer 412, a demand response (DR) layer 414, a fault detection and diagnostics (FDD) layer 416, an integrated control layer 418, and a building subsystem integration layer 420. Layers 410-420 can be configured to receive inputs from building subsystems 428 and other data sources, determine optimal control actions for building subsystems 428 based on the inputs, generate control signals based on the optimal control actions, and provide the generated control signals to building subsystems 428. The following paragraphs describe some of the general functions performed by each of layers 410-420 in BMS 400.

Enterprise integration layer 410 can be configured to serve clients or local applications with information and services to support a variety of enterprise-level applications. For example, enterprise control applications 426 can be configured to provide subsystem-spanning control to a graphical user interface (GUI) or to any number of enterprise-level business applications (e.g., accounting systems, user identification systems, etc.). Enterprise control applications 426 may also or alternatively be configured to provide configuration GUIs for configuring BMS controller 366. In yet other embodiments, enterprise control applications 426 can work with layers 410-420 to optimize building performance (e.g., efficiency, energy use, comfort, or safety) based on inputs received at interface 407 and/or BMS interface 409.

Building subsystem integration layer 420 can be configured to manage communications between BMS controller 366 and building subsystems 428. For example, building subsystem integration layer 420 may receive sensor data and input signals from building subsystems 428 and provide output data and control signals to building subsystems 428. Building subsystem integration layer 420 may also be configured to manage communications between building subsystems 428. Building subsystem integration layer 420 translate communications (e.g., sensor data, input signals, output signals, etc.) across a plurality of multi-vendor/multi-protocol systems.

Demand response layer 414 can be configured to optimize resource usage (e.g., electricity use, natural gas use, water use, etc.) and/or the monetary cost of such resource usage in response to satisfy the demand of building 10. The optimization can be based on time-of-use prices, curtailment signals, energy availability, or other data received from utility providers, distributed energy generation systems 424, from energy storage 427 (e.g., hot TES 242, cold TES 244, etc.), or from other sources. Demand response layer 414 may receive inputs from other layers of BMS controller 366 (e.g., building subsystem integration layer 420, integrated control layer 418, etc.). The inputs received from other layers can include environmental or sensor inputs such as temperature, carbon dioxide levels, relative humidity levels, air quality sensor outputs, occupancy sensor outputs, room schedules, and the like. The inputs may also include inputs such as electrical use (e.g., expressed in kWh), thermal load measurements, pricing information, projected pricing, smoothed pricing, curtailment signals from utilities, and the like.

According to some embodiments, demand response layer 414 includes control logic for responding to the data and signals it receives. These responses can include communicating with the control algorithms in integrated control layer 418, changing control strategies, changing setpoints, or activating/deactivating building equipment or subsystems in a controlled manner. Demand response layer 414 may also include control logic configured to determine when to utilize stored energy. For example, demand response layer 414 may determine to begin using energy from energy storage 427 just prior to the beginning of a peak use hour.

In some embodiments, demand response layer 414 includes a control module configured to actively initiate control actions (e.g., automatically changing setpoints) which minimize energy costs based on one or more inputs representative of or based on demand (e.g., price, a curtailment signal, a demand level, etc.). In some embodiments, demand response layer 414 uses equipment models to determine an optimal set of control actions. The equipment models can include, for example, thermodynamic models describing the inputs, outputs, and/or functions performed by various sets of building equipment. Equipment models may represent collections of building equipment (e.g., subplants, chiller arrays, etc.) or individual devices (e.g., individual chillers, heaters, pumps, etc.).

Demand response layer 414 may further include or draw upon one or more demand response policy definitions (e.g., databases, XML, files, etc.). The policy definitions can be edited or adjusted by a user (e.g., via a graphical user interface) so that the control actions initiated in response to demand inputs can be tailored for the user's application, desired comfort level, particular building equipment, or based on other concerns. For example, the demand response policy definitions can specify which equipment can be turned on or off in response to particular demand inputs, how long a system or piece of equipment should be turned off, what setpoints can be changed, what the allowable set point adjustment range is, how long to hold a high demand setpoint before returning to a normally scheduled setpoint, how close to approach capacity limits, which equipment modes to utilize, the energy transfer rates (e.g., the maximum rate, an alarm rate, other rate boundary information, etc.) into and out of energy storage devices (e.g., thermal storage tanks, battery banks, etc.), and when to dispatch on-site generation of energy (e.g., via fuel cells, a motor generator set, etc.).

Integrated control layer 418 can be configured to use the data input or output of building subsystem integration layer 420 and/or demand response layer 414 to make control decisions. Due to the subsystem integration provided by building subsystem integration layer 420, integrated control layer 418 can integrate control activities of the subsystems 428 such that the subsystems 428 behave as a single integrated supersystem. In some embodiments, integrated control layer 418 includes control logic that uses inputs and outputs from a plurality of building subsystems to provide greater comfort and energy savings relative to the comfort and energy savings that separate subsystems could provide alone. For example, integrated control layer 418 can be configured to use an input from a first subsystem to make an energy-saving control decision for a second subsystem. Results of these decisions can be communicated back to building subsystem integration layer 420.

Integrated control layer 418 is shown to be logically below demand response layer 414. Integrated control layer 418 can be configured to enhance the effectiveness of demand response layer 414 by enabling building subsystems 428 and their respective control loops to be controlled in coordination with demand response layer 414. This configuration may advantageously reduce disruptive demand response behavior relative to conventional systems. For example, integrated control layer 418 can be configured to assure that a demand response-driven upward adjustment to the setpoint for chilled water temperature (or another component that directly or indirectly affects temperature) does not result in an increase in fan energy (or other energy used to cool a space) that would result in greater total building energy use than was saved at the chiller.

Integrated control layer 418 can be configured to provide feedback to demand response layer 414 so that demand response layer 414 checks that constraints (e.g., temperature, lighting levels, etc.) are properly maintained even while demanded load shedding is in progress. The constraints may also include setpoint or sensed boundaries relating to safety, equipment operating limits and performance, comfort, fire codes, electrical codes, energy codes, and the like. Integrated control layer 418 is also logically below fault detection and diagnostics layer 416 and automated measurement and validation layer 412. Integrated control layer 418 can be configured to provide calculated inputs (e.g., aggregations) to these higher levels based on outputs from more than one building subsystem.

Automated measurement and validation (AM&V) layer 412 can be configured to verify that control strategies commanded by integrated control layer 418 or demand response layer 414 are working properly (e.g., using data aggregated by AM&V layer 412, integrated control layer 418, building subsystem integration layer 420, FDD layer 416, or otherwise). The calculations made by AM&V layer 412 can be based on building system energy models and/or equipment models for individual BMS devices or subsystems. For example, AM&V layer 412 may compare a model-predicted output with an actual output from building subsystems 428 to determine an accuracy of the model.

Fault detection and diagnostics (FDD) layer 416 can be configured to provide on-going fault detection for building subsystems 428, building subsystem devices (i.e., building equipment), and control algorithms used by demand response layer 414 and integrated control layer 418. FDD layer 416 may receive data inputs from integrated control layer 418, directly from one or more building subsystems or devices, or from another data source. FDD layer 416 may automatically diagnose and respond to detected faults. The responses to detected or diagnosed faults can include providing an alert message to a user, a maintenance scheduling system, or a control algorithm configured to attempt to repair the fault or to work-around the fault.

FDD layer 416 can be configured to output a specific identification of the faulty component or cause of the fault (e.g., loose damper linkage) using detailed subsystem inputs available at building subsystem integration layer 420. In other exemplary embodiments, FDD layer 416 is configured to provide “fault” events to integrated control layer 418 which executes control strategies and policies in response to the received fault events. According to some embodiments, FDD layer 416 (or a policy executed by an integrated control engine or business rules engine) may shut-down systems or direct control activities around faulty devices or systems to reduce energy waste, extend equipment life, or assure proper control response.

FDD layer 416 can be configured to store or access a variety of different system data stores (or data points for live data). FDD layer 416 may use some content of the data stores to identify faults at the equipment level (e.g., specific chiller, specific AHU, specific terminal unit, etc.) and other content to identify faults at component or subsystem levels. For example, building subsystems 428 may generate temporal (i.e., time-series) data indicating the performance of BMS 400 and the various components thereof. The data generated by building subsystems 428 can include measured or calculated values that exhibit statistical characteristics and provide information about how the corresponding system or process (e.g., a temperature control process, a flow control process, etc.) is performing in terms of error from its setpoint. These processes can be examined by FDD layer 416 to expose when the system begins to degrade in performance and alert a user to repair the fault before it becomes more severe.

### Pathogen Detection System

Referring now to FIG. 5, a pathogen detection system 500 (sometimes referred to herein as “integrated biosensing system”) for building 10 is shown, according to some embodiments. Any of the functionality of pathogen detection system 500 as described herein may be implemented in any of the BMS as described in greater detail above with reference to FIGS. 1-4. As shown in FIG. 5, the pathogen detection system 500 includes multiple pathogen detectors 504 that are positioned throughout building 10. The pathogen detectors 504 (e.g., sensors, sensing elements, etc.) can be configured to detect a presence and/or a type of pathogen (e.g., an airborne pathogen, DNA, RNA, etc.) within the building 10. The pathogen detectors 504 can be positioned in different zones 506 of the building, or may be integrated within components of the HVAC system 100 that serves the building 10. It should be understood that while the detection controller 502 as described herein obtains pathogen detection data from pathogen detectors 504, the detection controller 502 can also obtain temperature, humidity, occupancy, carbon dioxide, indoor air quality, etc., or any other sensor data from sensors within the building 10 and use such sensor data to determine appropriate responses, identify high risk areas, generate dashboards, perform control operations, provide notifications to occupants, etc. It should also be understood that, while the illustrated implementation includes multiple pathogen detectors 504 positioned in different zones 506 or different locations within or near the building, in some implementations, only one pathogen detector may be used, or less or more pathogen detectors than are illustrated may be used. The pathogen detectors 504 can be communicably coupled via a wired connection with the detection controller 502, or wirelessly (e.g., by communicating with the detection controller 502 via Bluetooth, LoRa, Zigbee, via cellular communications, a wireless network, a building Wi-Fi network, etc.).

For example, the pathogen detectors 504 can be configured to identify RNA and/or DNA of a pathogen of interest (e.g., influenza, COVID-19 or a particular strain thereof, Ebola, etc.). In some embodiments, pathogen detectors 504 (e.g., 504a-c) may collect air samples of different particulate matter or pathogens on a sample strip of the pathogen detectors 504 that can be analyzed to determine a presence of a pathogen of interest (or a presence of one or more pathogens of interest). In some embodiments, pathogen detectors 504 (e.g., 504d) may collect wastewater or sewage samples of different matter or pathogens based on coupling a pipe or conduit that is configured to receive sewage from or within a building, that can be analyzed to determine a presence of a pathogen of interest (or a presence of one or more pathogens of interest). It should be understood at least some of the pathogen detectors 504 may collect various pathogens or matter via different collections techniques or mechanism using different types of collectors. For example, some of the collectors may be different types of collectors that do not collect air samples but collet samples in some other form, such as sewage-based, surface-based, etc. In some embodiments, the pathogen detectors 504 are configured to provide results to a detection controller 502 of the pathogen detection system 500 after a processing of the samples obtained is completed. The processing of the pathogen detectors 504 may extend over a 6-18 hour period. In some embodiments, the processing of the pathogen detectors 504 may extend over a 24 hour period. In some embodiments, the processing of the pathogen detectors 504 may extend over a 1 hour period or a 30 minute period. Results of the processing of the pathogen detectors 504 can be provided to the detection controller 502 as detection signals when available. The pathogen detectors 504 can include processing circuitry, various processors, memory, etc., for performing the processing of the samples. In some embodiments, the pathogen detectors 504 are configured to provide the results of the processing to the detection controller 502 in real-time. In some embodiments, pathogen detector 504d is a different type of detector then 504a-c/n. In some embodiments, the pathogen detectors 504 utilize techniques for detecting pathogens, infectious quanta, etc., as described in greater detail in PCT/US2021/062444, filed Dec. 8, 2021, the entire disclosure of which is incorporated by reference herein.

The pathogen detectors 504 can be configured to detect a single type of pathogen, and may output an indicator of detection and the type of pathogen detected to the detection controller 502, according to some embodiments. In some embodiments, the pathogen detectors 504 are configured to detect multiple types of pathogens, such as by including multiple types of pathogen detectors or sensors on a single pathogen detector 504. In some embodiments, the pathogen detectors 504 are re-configurable to detect different types of pathogens such as through a module interface that allows for selective attachment or coupling of various adapters or sensors, each configured to allow the pathogen detector 504 to detect a different type of pathogen. The pathogen detectors 504 can include any combination of single-pathogen detectors, multiple pathogen detectors, modular pathogen detectors, etc.

The pathogen detectors 504 can be positioned throughout the building 10 at different locations. For example, a first pathogen detector 504a may be positioned in a zone 506a proximate an entrance of the building 10. Similarly, a second pathogen detector 504b can be positioned at a zone 506b within an AHU return line (e.g., return piping of AHU 106). For example, the AHU 106 may include a collection pipe with holes directed into air streamlines to allow the pathogen detector 504b to obtain a representative sample of return air from a zone of the building 10). A third pathogen detector 504c may be positioned in a zone 506c of the building 10 where responsive actions can be taken (e.g., alerts, disinfection operations, pathogen reduction control, etc.). A fourth pathogen detector 504d can be positioned in a sewage line 506d of the building 10. The pathogen detector 504d (or any of the other pathogen detectors 504 described herein) can use quick polymerase chain reaction (qPCR) functionality to detect the presence of a pathogen. In some embodiments, pathogen detector 504d can be a biosensor and/or transducers configured to detect pathogens in sewage, wastewater, sludge. For example, the pathogen detector 504d can be, but is not limited to, a fluorescent peptides sensor, plasmonic ELISA sensor, impedimetric sensor, voltammetric and amperometric proteins sensors, mechanical sensor, electrochemical PCR-free Mycobacterium tuberculosis (MTB) genomic sensor, other genomic sensors, fluorescent peptides sensors, fluorescent array sensor, evanescent wave fiber-optic sensor, laser cytometry sensor, electrochemical and mass sensitive DNA/antibodies sensor, optical sensor, a combination of sensors, etc. For example, a genomic sensor can collect and process samples using cellular-level molecular biology to detect pathogens.

An nth pathogen detector 504n can be positioned in a zone 506n that is a key focus space (e.g., an area of the building 10 where pathogen detection may be likely to occur, a highly populated or high traffic area of the building 10 such as a restroom of the building 10, a choke point of the building 10 where occupant density is high, an isolated room or zone of building 10, etc.). In some embodiments, the detectors 504 are configured to use electrochemical means to detect a pathogen or a virus. In some embodiments, the pathogen detectors 504 are configured to detect pathogens or viruses on a surface. In some embodiments, the pathogen detectors 504 are configured to detect airborne pathogens or viruses. In some embodiments, the pathogen detectors are configured to sample particles in the air of the building 10 which is then fed into a qPCR detection apparatus. In some embodiments, the pathogen detectors 504 are or include Coriolis micro-microbial air samplers that are configured to detect a presence of a pathogen. In some embodiments, the pathogen detectors 504 are configured to use real-time PCR detection techniques to detect a presence of a pathogen or a virus.

In some embodiments, at least one pathogen detector 504 is positioned at one or more entrances, exits, or access points of the building 10. In some embodiments, at least one pathogen detector 504 is positioned at every entry area where employees typically badge in (e.g., scan a badge at a card reader) of the building 10 or a facility. The pathogen detectors 504 may be configured to periodically sample air in the entry area at periodic intervals (e.g., every 30 minutes). In some embodiments, a time at which each air sample is obtained is also recorded (e.g., a time-stamp). In some embodiments, the detection controller 502 is configured to communicate with the pathogen detectors 504 and also with an access or security system of the building 10 that includes the card reader where the employees or occupants scan their badges to access the building. The detection controller 502 can use the recorded times at which the air samples are obtained by the pathogen detectors (and therefore the detection results) in combination with a number of badge logs scanned by the card reader at the same time, or within a same time window. For example, if the air sample obtained by the pathogen detectors 504 at a particular entrance from 9:30 AM to 10 AM indicates pathogen detection, the number of badge logs may be correlated to the air samples. The detection controller 502 can then identify identities of different occupants who entered the building between the times from 9:30 AM to 10 AM (when the pathogen is detected). The detection controller 502 may operate as described herein to request that these individuals be tested (e.g., to determine if the individuals are infected or carrying the pathogen).

In some embodiments, the periodic interval is adjusted in real-time based on a number of employees entering the building 10. For example, the detection controller 502 may identify, based on a number of badge swipes at the entrance, in response to pressure sensors within a floor at an entrance, based on camera data, based on sensor data from a door sensor, etc., a traffic level through the entrance of the building 10 where one or more pathogen detectors 504 are located. If the traffic level indicates a high amount of traffic, the detection controller 502 may update the periodic interval or initiate pathogen detection so that detection results are obtained from the pathogen detectors 504 (e.g., changing the periodic interval from 30 minutes to 25 minutes, initiating the pathogen detectors 504 to begin sampling air based on the traffic level, etc.). Similarly, if the traffic level indicates a low amount of traffic, the detection controller 502 may update the periodic interval of the pathogen detectors 504 so that detection results are obtained from the pathogen detector 504 less frequently (e.g., changing the periodic interval from 30 minutes to 45 minutes) or may shut-off pathogen detection (e.g., shutting off operation of the pathogen detectors 504 based on the traffic level). In this way, the pathogen detection or operation of pathogen detectors 504 thereof may be triggered additionally or alternatively based on traffic level and/or based on a periodic interval. For example, the pathogen detectors 504 may obtain air samples in response to a predetermined number of people passing through the entry (e.g., every 20 individuals, every 50 individuals, etc.) instead of according to a periodic time interval.

It should be understood that in various embodiments, the pathogen detection system 500 can be configured to operate the pathogen detectors 504 to test for pathogens based on a time interval (e.g., a variable based time interval, a periodic interval, based on traffic level, etc.) and/or based on a number of detected people (e.g., also variable based) passing through the entrance, some combination thereof, or may switch between the two (e.g., periodically sampling and testing during low traffic level, and sampling based on a number of occupants that have entered the building during high traffic level or different times of day when traffic level is high). Advantageously, these techniques can facilitate better resolution of pathogen detection at busy times (e.g., before a shift starts in the building 10 when traffic is expected to be high), as well as to save expenses associated with operating the pathogen detectors 504 at lower traffic times.

In some embodiments, since most employees may enter the building 10 prior to a beginning of a shift, and there may be limited value in re-testing individuals as they leave and re-enter the building 10, the detection controller 502 and the pathogen detectors 504 can be configured to sample at times of day when shift changes are expected. In some embodiments, the detection controller 502 can use a scheduled occupancy (e.g., scheduled shifts) of the building 10 to determine when the shift changes are expected, or more generally, to determine when higher traffic will occur, and thereby decrease the periodic interval at which pathogens are detected to achieve a higher resolution of pathogen detection. For example, if the building 10 is a factory, the detection controller 502 and the pathogen detectors 504 may be configured to only obtain air samples at the beginning of a shift, or at known shift changes. Similarly, if the building 10 is an office building, the detection controller 502 and the pathogen detectors 504 may focus on obtaining air samplings in the morning (e.g., 7 AM to 10 AM).

In this way, the locations of the pathogen detectors 504 throughout building 10 can be designed to detect pathogen presence in an intelligent manner, based on areas of interest, or in a zone-by-zone manner so that a location of the pathogen can be detected in addition to a presence of a pathogen. In some embodiments, one or more of the pathogen detectors 504 are positioned on a mobile or portable device that is configured to translate or travel throughout the building 10. For example, the portable device may be configured to move through the building 10, while providing the detection controller 502 with real-time pathogen detection results, and providing the detection controller 502 with a real-time location of the portable device in the building 10. In this way, the portable device may seek or “sniff” for pathogens throughout the building 10 and provide the detection controller 502 with detection results so that a location of a pathogen within the building 10 can be determined. The building 10 can be retrofit with the pathogen detectors 504 being placed in areas of interest, highly populated areas of the building, areas of the building where a pathogen would be likely to be detected, etc.

Detection controller 502 is configured to obtain the detection results from any of the pathogen detectors 504 when results are available from the pathogen detectors 504 (e.g., in a real-time basis, in near-real time, in 24 hour intervals, etc.). Detection controller 502 can obtain the detection results and analyze the detection results to identify if a pathogen is detected in the building 10, a type of pathogen that is detected in the building 10, and/or a location of the detected pathogen in the building 10. The detection controller 502 can be configured to use known locations of the different pathogen detectors 504 and generate appropriate data (e.g., commands, analytical data, control signals, alert data, etc.) for any of a messaging system 508, a control system 510, an analytics system 512, a monitoring system 514, one or more service application system 516, and/or an alert system 518, etc., to perform one or more responsive actions in response to detecting a presence of a pathogen in the building 10. In some embodiments, the detection controller 502 is also configured to generate and/or provide control signals to the HVAC system 100 of the building 10. In some embodiments, the detection controller 502 is configured to determine and provide informative data for the HVAC system 100 for use by the HVAC system 100 in determining control operations thereof. In some embodiments, the detection controller 502 provides different data to any of the messaging system 508, the control system 510, the analytics system 512, the monitoring system 514, the service application system 516, and/or the alert system 518 based on a type of pathogen that is detected, and/or a location of where in the building 10 the pathogen is detected. This may result in different responsive actions for the detection of different types of pathogens. Certain pathogens may result in the detection controller 502 initiating more drastic responsive actions (e.g., evacuation and/or closure of a space), while other pathogens may result in the detection controller 502 initiating less drastic responsive actions (e.g., user alerts, cleaning of space, etc.).

In some embodiments, the detection controller 502 is located on-site at building 10. In some embodiments, any of the systems 508-518 are located off-site (e.g., in a cloud computing system as part of a service). In some embodiments, the detection controller 502 is also located off-site (e.g., in a cloud computing system) and communicates with the pathogen detectors 504 to obtain detection results.

In some embodiments, the detection controller 502 is also configured to determine a magnitude of the pathogen detection based on the detection results provided by the pathogen detectors 504. The magnitude of the pathogen detection can result in different responsive action being performed, thereby achieving appropriate responsive actions for the magnitude of the pathogen detection. For example, a single detection of influenza may result in a lower magnitude response than multiple detections of a particular strain of COVID-19, which may result in different responsive actions. The magnitude of the pathogen detection can be based on any of a number of positive detections of a pathogen (as indicated by the detection results), a type of pathogen that is detected, a location where the pathogen is detected in the building 10, etc., or any combination thereof. For example, if a pathogen is detected in an isolated room that can be easily sealed off (e.g., a nursing station, a sick room, etc.), the magnitude of the pathogen detection (and therefore a magnitude of the responsive actions) may be lower than a magnitude of a pathogen detection where multiple detections of a particular strain of COVID-19 are detected at a highly populated area of the building 10. Advantageously, the known locations of the pathogen detectors 504, and known architecture of the building 10 can facilitate accurate determination of the magnitude of the pathogen detection. The detection controller 502 can also be configured to obtain sensor data from any occupant sensor (e.g., a motion detector, a camera, lighting statuses, etc.) throughout the building 10 to aid in determining if the pathogen detectors 504 indicate the detection of a pathogen in a highly populated area of the building 10. The detection controller 502 can be configured to obtain occupant data from any number of occupant sensors and use the occupant data to determine the magnitude of the pathogen detection (e.g., determining a higher magnitude of the pathogen detection if the pathogen is detected in an area which the occupant data indicates is highly or densely populated).

In some embodiments, the detection controller 502 is configured to receive pathogen data from a data provider 520. The data provider 520 can be a government or clinical database configured to provide seasonal pathogen data (e.g., current types of strains, etc.) and/or a number of local infections in the area as the pathogen data. The detection controller 502 may adjust pathogen monitoring schemes based on the pathogen data.

Referring still to FIG. 5, the detection controller 502 can be configured to operate the messaging system 508 or initiated one or more operations to be performed by the messaging system 508. For example, if the detection controller 502 determines, based on the detection results provided by the pathogen detectors 504, that a pathogen has been detected in the building 10, or in a particular area of the building 10, the detection controller 502 may initiate or operate the messaging system 508 to provide emails, alerts, text messages, automated phone calls, notifications via a mobile application, etc., to one or more occupants, employees, service technicians, etc., of the building 10 or of the particular area of the building 10 where the pathogen is detected. The messages generated by the messaging system 508 can include notifications, recommendations, or commands that the occupants, employees, service technicians, etc., stay at home and do not enter the building 10 due to a detection. The messaging system 508 can also generate messages for cleaning crews or employees of the building 10, notifying the cleaning crews or employees regarding a type of pathogen that has been detected in the building 10, and a location in the building 10 where the pathogen has been detected. The cleaning crews can then use this knowledge to perform appropriate cleaning actions at the location of the building 10 where the pathogen has been detected and/or to use appropriate cleaning procedures based on the type of pathogen detected in the location.

Referring still to FIG. 5, the detection controller 502 can be configured to operate the control system 510 and/or initiate one or more actions of the control system 510. The detection controller 502 may provide any of, or any combination of, the detection results, a magnitude of pathogen detection, a type of pathogen detected, a location in the building 10 where pathogen detection has occurred, a number of instances of the detected pathogen in the building 10, etc. The control system 510 can be configured to use any of the detection results, the magnitude of the pathogen detection, the type of pathogen detected, the location in the building 10 where pathogen detection has occurred, the number of instances of the detected pathogen in the building 10, etc., in a high level control logic application to determine when to activate and deactivate infection control sequences, and/or to target infection control sequences to provide infection control to the location in the building 10 where the pathogen is detected. In some embodiments, the infection control sequences include any of, or any combination of, drawing fresh outdoor air (e.g., increasing an air-intake fraction) to improve fresh air ventilation, operating one or more filtration devices (e.g., filtration devices positioned locally in the building 10, filtration devices positioned in the HVAC system 100 of the building 10, etc.), and/or operating one or more ultraviolet (UV) lights to provide infection control.

In some embodiments, the control system 510 is configured to use the magnitude of the pathogen detection as provided by the detection controller 502 to determine an appropriate infection control sequence. For example, a higher magnitude of the pathogen detection may result in the control system 510 implementing a more aggressive infection control sequence, whereas a lower magnitude of the pathogen detection may result in the control system 510 implementing a less aggressive infection control sequence. The control system 510 can also use the location of the detected pathogens to target initiation of the various infection control sequences to the location in the building 10 where the pathogens are detected. For example, the control system 510 can implement UV light operation for various air ducts of an AHU that provide or recirculate air to the location in the building 10 where the pathogens are detected. Advantageously, the location of the pathogen detection may facilitate improved energy costs associated with the infection control sequences (e.g., by not activating infection control devices for areas of the building where the pathogen is not detected). In some embodiments, the control system 510 is also configured to adjust access to the building 10. For example, the control system 510 may be a portion of a security system of the building 10 or may be integrated within the security system of the building 10. The control system 510 can prevent additional occupants from entering zones or locations in the building 10 where the pathogen is detected. In some embodiments, the control system 510 is configured to use pathogen detection data obtained by the pathogen detectors 504 and/or any outputs of the detection controller 502 as inputs to, or to train models of the systems and methods described in greater detail in U.S. application Ser. No. 16/927,759, filed Jul. 13, 2020, the entire disclosure of which is incorporated by reference herein.

Referring still to FIG. 5, the detection controller 502 is configured to provide outputs to the analytics system 512, according to some embodiments. In some embodiments, the outputs provided from the detection controller 502 to the analytics system 512 are the same as the outputs provided by the detection controller 502 to the control system 510. The analytics system 512 is configured to use feedback regarding actually measured pathogens (e.g., the detection results) to validate and/or improve one or more prediction models (e.g., a Wells-Riley based prediction model such as for predicting concentration of infectious quanta). In some embodiments, the analytics system 512 can include a predictive model that is configured to combine both a deterministic prediction portion and a stochastic correction. The deterministic prediction portion can be based on various equations (e.g., the Wells-Riley equation), and the stochastic correction can be adjusted, generated, determined, updated, etc., based on the detection results provided to the analytics system 512 by the detection controller 502 to improve an accuracy of the predictive model. The stochastic correction may be an adaptive portion of the predictive model. The predictive model can be updated and provided to the control system 510 for use in initiating the infection control sequences. In some embodiments, the outputs provided by the detection controller 502 (e.g., any of the data gathered by the detection controller 502 from the detectors 504, or any of the responses performed and subsequently obtained data) may be used to calibrate, update, or be any other input to any of the models described in greater detail with reference to PCT/US2020/041845, filed Jul. 13, 2020, the entire disclosure of which is incorporated by reference herein.

In some embodiments, the analytics system 512 is configured to integrate with the security system of the building 10. For example, the analytics system 512 may use the location of the detected pathogens to deny entry access of the building 10 by additional occupants, to isolate zones or rooms where the pathogens are detected, and/or to initiate contact tracing before an individual is contagious.

In some embodiments, the analytics system 512 is configured to use the outputs of the detection controller 502 to determine if an “all-clear” condition has been met (e.g., to determine if the pathogen is no longer detected in the building 10). The analytics system 512 can continually receive the outputs of the detection controller 502 (e.g., the detection results) and if the outputs indicate that no pathogens, or a particular type of pathogen, has/have not been detected for a predetermined amount of time, the analytics system 512 may determine that the pathogen is no longer present in the building 10. The analytics system 512 may determine that the pathogen is no longer present in the building and can determine that the “all-clear” condition has been met. The analytics system 512 can function in cooperation with the messaging system 508 to send messages to various occupants or employees that the building 10 can once again be accessed in response to determining that the “all-clear” condition has been met. In some embodiments, the analytics system 512 functions in cooperation with the control system 510 to deactivate the infection control sequences, thereby conserving energy usage when the infection control sequences are not necessary. The “all-clear” condition may indicate that the building 10, or a space, zone, or area of the building 10 where a pathogen has been detected can be re-occupied.

In some embodiments, the analytics system 512 is configured to correlate individual or group identification numbers with an RNA or DNA sample of the detected pathogen to rapidly identify which occupants of the building 10 are carrying the pathogen. In some embodiments, the analytics system 512 only has access to the identification numbers if an occupant or individual opts to allow the analytics system 512 with such access.

Referring still to FIG. 5, the detection controller 502 is configured to provide the outputs to the monitoring system 514, according to some embodiments. In some embodiments, the monitoring system 514 includes, or is in communication with, one or more display devices, notification systems, etc. The monitoring system 514 may be a back-end monitoring system for an administrator of the building 10. In some embodiments, the monitoring system 514 communicates with any of the messaging system 508, the control system 510, the analytics system 512, the service application system 516, the alert system 518, the HVAC system 100, a BMS of the building 10, etc., so that the monitoring system 514 can obtain operational data thereof. In some embodiments, the monitoring system 514 is configured to generate dashboards, user-interfaces, graphical user interfaces, graphs, charts, diagrams, tabular data, etc., of any of the messaging system 508, the control system 510, the analytics system 512, the service application system 516, the alert system 518, the HVAC system 100, the BMS of building 10, and/or the detection controller 502 based on operational data, sensor data, analytic data, etc., thereof. For example, the monitoring system 514 can generate a dashboard that demonstrates what areas in the building 10 are currently or have previously undergone infection control, what type of pathogen is detected in any areas of the building, time series-data of pathogen detection in building 10, etc. In some embodiments, the monitoring system 514 is configured to use any of the data obtained by or determined by the detection controller 502 in combination with the techniques as described in U.S. application Ser. No. 16/927,281, filed Jul. 13, 2020, to generate visualizations or dashboards, the entire disclosure of which is incorporated by reference herein.

Referring still to FIG. 5, the detection controller 502 is configured to provide the outputs to the service application system 516, according to some embodiments. In some embodiments, the service application system 516 is configured to identify sales opportunities based on the outputs of the detection controller 502. For example, the service application system 516 can identify sales opportunities (e.g., service opportunities) such as dirty coils, dirty or slimy condensate pans, dirty filters, mold, excessive particulate matter or dust, etc. The service application system 516 can initiate a service (e.g., scheduling, contracting, etc.) to address to the different sales opportunities, according to some embodiments. In some embodiments, samples from the services are provided to a lab (e.g., mailed to a lab) for baseline assessment. When future services are scheduled and implemented, the service application system 516 can validate effectiveness of mitigation solutions performed at the building 10 relative to the baseline assessment using lab results of subsequently obtained samples. The service application system 516 can also optimize resource dispatching. For example, the service application system 516 can prompt technicians, work crews, individuals, etc., with proper skills, training, and equipment to address different identified sales opportunities (e.g., to clean filters of the HVAC system 100, to replace faulty UV lights, etc.). In some embodiments, the service application system 516 is configured to perform an optimization to determine optimal scheduling of work crews or technicians to address the different sales opportunities.

In some embodiments, the outputs of the detection controller 502 are used by various miscellaneous systems or to perform miscellaneous responsive actions. For example, the outputs of the detection controller 502 can be used to determine if infection control should be included in action priority (e.g., codes, standards, etc.). In this way, infection control may be prioritized appropriately (e.g., as a top priority) amongst other priorities such as energy efficiency, convenience, occupant comfort, etc. The systems and methods described herein can also facilitate economic advantages such as reducing insurance premiums due to the active monitoring of pathogens and the active mitigation of pathogens in the building 10.

Referring still to FIG. 5, the detection controller 502 is configured to provide the outputs to the alert system 518, according to some embodiments. In some embodiments, the alert system 518 is a controller or a control device of an alert system of the building 10. The alert system can include various aural alert devices (e.g., alarms, speakers, sirens, etc.) positioned throughout the building 10 and/or visual alert devices (e.g., warning lights, display screens, etc.) positioned throughout the building 10. The detection controller 502 can operate the alert system 518 in response to detecting a pathogen, a particular type of pathogen, a number of pathogen detection instances greater than a threshold, a magnitude of the pathogen detection being above a threshold magnitude, etc. In some embodiments, the detection controller 502 is configured to operate the alert system 518 to provide visual and/or aural alerts in a location where the pathogen is detected. In this way, the alert system 518 can use the location of the detected pathogens to provide targeted alerts to occupants of the building 10. In some embodiments, the alert system 518 performs the responsive action by providing a visual alert regarding policy changes in the building 10 (e.g., prompting occupants to wear masks, prompting occupants from a location where a pathogen is detected to get tested, prompting occupants to ensure social distancing practices are followed, etc.).

Referring still to FIG. 5, the various systems (e.g., the messaging system 508, the control system 510, the analytics system 512, the monitoring system 514, the service application system 516, the alert system 518, etc.) may be components of the detection controller 502, or may be components of other processing circuitry (e.g., distributed processing circuitry, cloud computing systems, etc.) It should be understood that while detection controller 502 is described herein as determining responsive actions for each of the messaging system 508, the control system 510, the analytics system 512, the monitoring system 514, the service application system 516, the alert system 518, and the HVAC system 100, the detection controller 502 may, in some embodiments, be configured to only determine responsive actions for one or more of the messaging system 508, the control system 510, the analytics system 512, the monitoring system 514, the service application system 516, the alert system 518, etc.

Referring to the pathogen detectors 504 and detection controller 502 in more detail. The pathogen detection system 500 can include multiple pathogen detectors 504 that are position throughout building 10. In some embodiments, pathogen detector 504d may be a biological sensor positioned in or near a sewage or wastewater line or output of building 10. For example, pathogen detector 504d can include a transducer and a biological element (e.g., enzyme, cells, aptamers, DNA, antibody, nucleic acid). In particular, the biological element can interact with the analyte (e.g., pathogen) being tested and the biological response is converted into an electrical signal by the transducer. The electrical signal can be outputted to the detection controller 502, according to some embodiments. In another example, pathogen detector 504d can use quick polymerase chain reaction (qPCR) functionality to detect the presence of a pathogen or virus. In various embodiments, the pathogen detectors 504 utilize techniques for detecting pathogens, infectious quanta, etc., as described in greater detail in PCT/US2021/062444, filed Dec. 8, 2021, the entire disclosure of which is incorporated by reference herein.

Pathogen detectors 504 can include processing circuitry, various processors, memory, etc., for performing the processing of the samples. One or more of the samples (e.g., each sample) can be time stamped and stored for future analysis. In some embodiments, samples can be time stamped and/or location-tagged by detection controller 502. In various embodiments, the samples can be tagged with an identified of a location from which the sample is received or with an identifier of a pathogen detector from which can be used to determine a location by detection controller 502. For example, the biological sensor (e.g., 504d) can collect daily samples of wastewater to monitor sewage and the biological element can analyze the samples for one or more viruses (e.g., coronaviruses (e.g., COVID-19, SARS-CoV-2, SARS-CoV-3), influenza, hepatitis, HIV, chickenpox, etc.), one or more bacteria (e.g., E. coli, listeria, salmonella, etc.), and/or any other pathogen. In various embodiments, if the biological sensor (504d) detects a sample is positive for one or more viruses, bacteria, or pathogen, the processing circuitry may take one or more actions in response. In some such implementations, the processing circuitry of the biological sensor may collect additional samples to re-confirm the presence of the particular virus or bacteria. In some embodiments, the processing circuit (e.g., in parallel) can inform the detection controller 502 of the positive test (e.g., via output detection data including a detected presence of a pathogen).

In some implementations, the detection controller 502 can be configured to request additional samples (e.g., additional output detection data) from other pathogen detectors (e.g., 504a, 504b, 504c) throughout building 10. For example, the detection controller 502 can request air samples from pathogen detectors 504a and 504b, surface samples from pathogen detectors 504c, and sewage samples form pathogen detectors 504d. In particular, pathogen detectors 504a and 504b can be configured to detect airborne pathogens or viruses and pathogen detector 504c can be configured to detect pathogens or viruses on a surface (e.g., performing swab analysis via swabs that are used on surfaces to detect pathogens or viruses).

In some embodiments, pathogen detectors 504a and 504b can be configured to detect sewage. As shown, the additional pathogen detectors 504a, 504b, 504c, 504n can begin, in real-time (or near real-time), detecting pathogens (i.e., turned on/woke from sleep) in response to the biological sensor (e.g., 504d) identifying a sample with a pathogen or virus and communicating the determination to detection controller 502. In another example, detection controller 502 can be configured to perform analysis of the historical samples (and data collected) already collected by pathogen detectors for the past time period (e.g., 30 minutes, 2 hours, 3 days, 7 days, 2 months, etc.). In yet another example, detection controller 502 can be configured to request additional samples from the pathogen detectors and perform analysis of historical samples. As such, it should be understood that detection controller 502 can increase or decrease frequency of sampling and/or analysis based on various previous analysis, previous collected samples, and/or other parameters (e.g., building occupancy, pathogen community data, etc.)

In some embodiments, swab analysis can be performed by pathogen detector 504d in response to receiving surface samples collected by pathogen detector 504c, or other pathogen detectors within building 10. In particular, pathogen detector 504d can be configured to detect pathogen in wastewater (e.g., sewage) as well as receive samples from other pathogen detectors 504 to detect airborne pathogens or surface pathogens. The received samples and collected samples can be tested and communicated to detection controller 502. In some embodiments, pathogen detectors 504 can be communicably coupled via a wired or wireless connection with other pathogen detectors 504. Advantageously, the integrated pathogen detector solution that provides multiple types of pathogen detectors across building 10 may facilitate reduced costs (e.g., energy) associated with the infection control sequences (e.g., by not activating infection control devices for areas of the building where the pathogen is not detected) and increased efficiency in identifying particular sources of pathogen spread within building 10.

In various embodiments, the detection controller 502 can provide outputs to the analytics system 512 based on the samples collected and/or tested by pathogen detectors 504. The analytics system 512 can use the data (e.g., samples, positives, etc.) collected by the multiple pathogen detectors 504 to identify, in real-time or within a short period of time (e.g., 1 minute, 1 hour, 3 hours, 6 hours) one or more locations (or areas or zones) within building 10 of the positive tests (e.g., infections) source. For example, room 113 and room 115 of building 10 could be identified as the source of positive tests. In the following example, the determination can be based on a plurality of pathogen detectors collecting different samples (e.g., sewage sample, surface sample, airborne sample). In various implementations, the detection controller 502 can detect presence of pathogens and/or locations of the detection. In some embodiments, messaging system 508 can if the detection controller 502 determines, based on the detection results provided by the pathogen detectors 504, that a pathogen has been detected in building 10, or in a particular area of the building 10, the detection controller 502 may initiate or operate the messaging system 508 to provide emails, alerts, text messages, automated phone calls, notifications via a mobile application, etc., to one or more occupants, employees, service technicians, etc., of the building 10 or of the particular area of the building 10 where the pathogen is detected.

Accordingly, the pathogen detectors 504 positioned throughout the building to detect pathogens or viruses that are airborne, on surfaces, or in wastewater. Once one or more locations (or areas or zones) of building 10 are identified, one or more mitigation steps can be taken by HVAC system 100. Example mitigation steps that can be taken by HVAC system 100, according to various embodiments, are described with further reference to U.S. Provisional Application No. 63/307,981, filed Feb. 8, 2022, the entire disclosure of which is incorporated by reference herein. Additionally, detection controller 502 may be configured to continue to request (e.g., every hour, every 3 hours, every day) samples from pathogen detectors 504 until no samples are positives for a specified period of time (e.g., 12 hours, 3 days, 7 days, 10 days). In some embodiments, as additional positive tests are identified, the analytics system 512 can continue to identify and update, in real-time (or near real-time), locations (or areas or zones) of the positives within building 10 based on the pathogen detectors 504.

In various embodiments, the pathogen detectors 504 may be configured to sense the presence or absence of a pathogen by one or both of collecting samples in a particular location/zone of the building and having the samples collected and moved to a different portion of the building or off-premises for testing, and/or by testing the samples in-place where they are taken (e.g., using electronic circuitry and/or other features of the pathogen detectors 504). For example, in some implementations, the pathogen detector 504d may take a sample of sewage in a line of the building or at an outlet of the building and apply the sewage sample to a test unit (e.g., test strip) that is removable for testing at a different location. In some implementations, the pathogen detector 504d may be configured to test the sample in place, i.e., at or near where the sample was collected. For example, the pathogen detector 504d may be or include an electronic sampling device that takes a sample from the sewage, applies it to a sampling surface or unit, and tests the sample to determine the presence or absence of a pathogen or other substance in-zone or in-location. In some such embodiments, the pathogen detector 504d may include a transceiver (e.g., a wired or wireless communication device) configured to transmit a signal to one or more external devices indicating a presence and/or absence of detection of the pathogen in one or more samples.

In some such embodiments, the transmitted signal may be used by the external device(s) to perform one or more actions in response, such as the actions described elsewhere in the present disclosure. For example, in some implementations, responsive to receiving a signal indicating a detected presence of the pathogen by the pathogen detector 504d, an external device such as the detection controller 502 may take one or more actions to assess the potential spread of the pathogen (e.g., cause additional samples from other detectors to be sampled), limit the spread of the pathogen (e.g., activate additional pathogen reduction methods such as increased filtration in HVAC equipment, additional sanitization measures such as disinfectant light or substance application, etc.), or activate additional detection mechanisms (e.g., cause additional pathogen detectors 504 to take samples, such that the other detectors only take or test samples when pathogen detector 504d has a positive test or take or test samples more frequently in response to the positive test by pathogen detector 504d). In some implementations, some of the pathogen detectors 504 may be configured to have removable samples to be tested at a different location and others may be configured to test the samples where they are sampled.

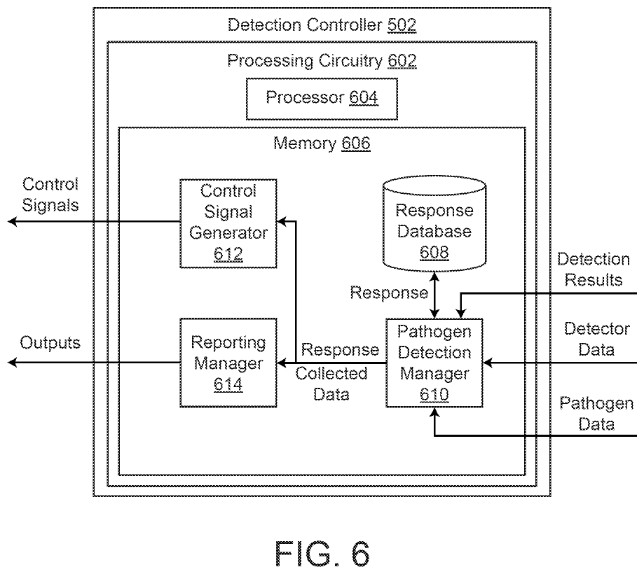

Referring particularly to FIG. 6, the detection controller 502 is shown in greater detail, according to some embodiments. The detection controller 502 is shown to include processing circuitry 602 including a processor 604 and memory 606. Processing circuitry 602 can be communicably connected to a communications interface such that processing circuitry 602 and the various components thereof can send and receive data via the communications interface. Processor 604 can be implemented as a general purpose processor, an application specific integrated circuit (ASIC), one or more field programmable gate arrays (FPGAs), a group of processing components, or other suitable electronic processing components.

Memory 606 (e.g., memory, memory unit, storage device, etc.) can include one or more devices (e.g., RAM, ROM, Flash memory, hard disk storage, etc.) for storing data and/or computer code for completing or facilitating the various processes, layers and modules described in the present application. Memory 606 can be or include volatile memory or non-volatile memory. Memory 606 can include database components, object code components, script components, or any other type of information structure for supporting the various activities and information structures described in the present application. According to some embodiments, memory 606 is communicably connected to processor 604 via processing circuitry 602 and includes computer code for executing (e.g., by processing circuitry 602 and/or processor 604) one or more processes described herein.

As shown in FIG. 6, memory 606 includes a response database 608, a pathogen detection manager 610, a control signal generator 612, and a reporting manager 614, according to some embodiments. The pathogen detection manager 610 is configured to receive detection results and pathogen data (e.g., airborne samples, surface samples, sewage samples) from the pathogen detectors 504, according to some embodiments. In some embodiments, the pathogen detection manager 610 is also configured to obtain detector data from each of the pathogen detectors 504. The detector data may include information regarding a type of detector, a location of the detector in the building 10, a model of the detector, installation data of the detector, measurement errors or uncertainties, control parameters, configuration data, etc. In some embodiments, the pathogen detection manager 610 is configured to determine or select an appropriate response from the response database 608 based on the detection results, pathogen data, and the detector data. For example, the pathogen detection manager 610 can be configured to determine a magnitude of the detection based on the detector data, according to some embodiments. In another example, the pathogen detection manager 610 can be configured to determine a location of pathogen spread and/or amount of pathogen spread based on the detector data, according to some embodiments. In some embodiments, the detector data also includes a magnitude or level of indication of the detection at each of the detectors 504. The magnitude or level of indication of the detection can be a building-wide indication, a floor-wide indication, a zone-wide indication, a room-wide indication, etc., according to some embodiments. For example, a detector that is placed within a return air duct of the building 10 (e.g., detector 504b) that draws air from a zone including multiple rooms or sub-zones may report a zone-wide level of indication or magnitude, according to some embodiments. In another example, a detector that is placed within a sewage line of the building (e.g., detector 504d) that detects pathogens in sewage exiting the building 10 may report a building-wide level of indication or magnitude, according to some embodiments. In yet another example, a detector that is placed within a single room (e.g., detector 504a) may report a room-wide level of indication or magnitude, according to some embodiments. In this way, the positioning and configuration of the detectors can indicate a degree of locality (e.g., a spatialization) of pathogen detection in the building 10. In yet another example, a detector that is coupled to a sewage line or output (e.g., detector 504d) may report an area (e.g., particular bathroom) or building indication of a particular pathogen, according to some embodiments. The pathogen detection manager 610 may determine the degree of locality based on the detector data for each of the detectors 504, or may receive the degree of locality from each of the detectors 504.

The pathogen detection manager 610 can use any of the detection results (e.g., a general location of detection of a pathogen, a number of instances of detection), the detector data (e.g., the degree of locality of each of the detectors 504), and the pathogen data (e.g., a type of pathogen detected) to determine or select a response (e.g., a responsive action) from the response database 608. The pathogen detection manager 610 can detect a presence of pathogens and/or locations of the detection, according to some embodiments. The pathogen detection manager 610 can select both a magnitude of the response and a locale magnitude of the response, according to some embodiments. In some embodiments, the magnitude of the response is a metric that is responsive to a severity of outbreak within the building. The magnitude of the response may be quantified based on a rated degree of intrusiveness to occupants of the building 10, an expected amount of energy consumption required to perform the response (e.g., using UV lights to reduce infection risks may require more energy consumption than increasing fresh air intake to reduce infection risks and thereby have a higher magnitude), and/or an expected infection risk reduction of performing the response. In some embodiments, the locale magnitude of the response indicates how many locations of the building 10 the response should affect. For example, the locale magnitude may indicate if the response should affect the entire building 10, a single room of the building 10, a zone of the building 10, multiple zones or rooms of the building 10, etc. In some embodiments, the locale magnitude is determined by the pathogen detection manager 610 based on the level of indication or magnitude of the detection by the detectors 504. For example, if a detector that monitors sewage exiting the entire building 10 detects of a pathogen, the pathogen detection manager 610 may determine that the response should affect the entire building 10 (e.g., changing a policy for the entire building 10, implementing a control sequence to affect the entire building 10, alerting all occupants of the entire building 10, etc.). Similarly, if a detector that monitors a return air in a duct that draws air from several zones of the building 10 detects a pathogen, the pathogen detection manager 610 may determine that the response should affect the several zones of the building 10 where the pathogen is detected.

In some embodiments, the pathogen detection manager 610 is configured to approximate a location or a range of locations in the building 10 based on the detector data and the detection results in which a particular pathogen may be present. The pathogen detection manager 610 can select or determine the response from the response database 608 and apply the response to the location or range of locations (e.g., initiate the response so that the response is performed) in response to the detection of the particular pathogen, according to some embodiments. For example, the detection manager 610 can determine a location of one or more pathogens based on collected samples from one or more pathogen detector 504 (e.g., a-d). In some embodiments, the pathogen detection manager 610 is configured to determine the magnitude of the response based on the pathogen data (e.g., based on the type of pathogen detected) and based on a number of instances of the pathogen being detected in the building 10. For example, if a particular pathogen such as COVID-19 is detected, the magnitude of the response may be relatively high, whereas if a different pathogen is detected, the magnitude of the response may be lower. Similarly, if a single instance of the pathogen is detected, the magnitude of the response may be lower than if multiple instances of the pathogen are detected. In this way, the response can have two properties—(i) a magnitude of the response itself that is quantified in terms of (a) invasiveness to the occupants of the building 10 (e.g., closing off an entire floor may be more invasive than prompting building staff to disinfect a particular area, requiring occupants of the building 10 to wear masks may be less invasive than prompting occupants to seek testing for infection but more invasive than initiating a control sequence to reduce infection risks), (b) expected energy or monetary expenditure required to perform the response, and/or (c) expected infection risk reduction resulting from performing the response, and (ii) a locale magnitude that quantifies a number of areas or spaces of the building 10 that the response is applied to (e.g., to a single floor, to a single room, to the entire building, to a zone of multiple areas of the building 10, etc.). The magnitude of the response may be determined or selected by the pathogen detection manager 610 based on the type of pathogen detected and/or a number of instances of detection of the pathogen, according to some embodiments. In some embodiments, the locale magnitude of the response is determined by the pathogen detection manager 610 based on which of the detectors 504 reports the detected pathogen, and which areas of the building 10 the detected pathogen may be present in based on the configuration of the detectors 504.

In some embodiments, the pathogen detection manager 610 is configured to provide the response and/or any of the collected data (e.g., from the detectors 504, sewage sample, airborne sample, surface sample) to the reporting manager 614 and/or the control signal generator 612. The control signal generator 612 can generate control signals for equipment of the building 10 to implement the response, according to some embodiments. In some embodiments, the control signals are provided to the HVAC system 100 or to an infection risk reduction system so that the HVAC system 100 or the infection risk reduction system can operate to reduce infection risk inside of the building 10 by increasing fresh air intake, operating UV lights, operating in-zone filtration devices, etc. In some embodiments, the reporting manager 614 is configured to provide any of the response, the magnitude of the response, the locale magnitude of the response, or the collected data to any of the messaging system 508, the control system 510, the analytics system 512, the monitoring system 514, the service application system 516, or the alert system 518 so that the systems 508-518 can perform their respective functions.

### Process

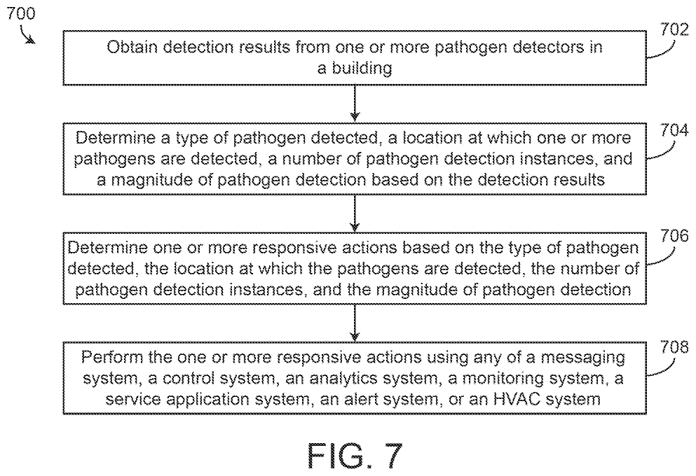

Referring to FIG. 7, a process 700 for performing pathogen detection for a building and performing one or more responsive actions is shown, according to some embodiments. The process 700 can be performed by the detection controller 502, and/or by the pathogen detection system 500 as described in greater detail above with reference to FIGS. 5-6. Process 700 includes steps 702-708, according to some embodiments. Process 700 can be performed using real-time or delayed pathogen detection results.

Process 700 includes obtaining detection results from one or more pathogen detectors in a building (step 702), according to some embodiments. The pathogen detectors can be pathogen detectors 504, according to some embodiments. In some embodiments, the pathogen detectors are or include static pathogen detectors (e.g., stationary and installed in a fixed location of the building). In some embodiments, the pathogen detectors are positioned at isolated rooms, entrances of the building, exits of the building, areas of the building with high expected occupancy density, etc. The pathogen detectors can also be positioned at areas of the building where pathogen detection is predicted to be high (e.g., in the bathroom, in a space of the building that is frequented by a large number of occupants, etc.). Locations of the pathogen detectors (e.g., floor, room, zone, etc.) may be known and can be used in steps 704-708, according to some embodiments. The pathogen detectors can also include a mobile unit including a pathogen detector that is configured to translate or move throughout the building, according to some embodiments. In some embodiments, step 702 is performed by the detection controller 502 and the pathogen detectors 504 as described in greater detail above with reference to FIGS. 5-6. Step 702 can also include obtaining seasonal infection or pathogen data for a region (e.g., a state, a country, a city, a country, a province, etc.) in which the building is located.

Process 700 includes determining a type of pathogen detected, a location at which one or more pathogens are detected, a number of pathogen detection instances, and a magnitude of pathogen detection based on the detection results (step 704), according to some embodiments. Step 704 can be performed by the detection controller 502, according to some embodiments. The type of pathogen detected may be an output of any of the pathogen detectors. For example, the type of pathogen may be a particular strain of a pathogen such as COVID 19, different strains of influenza, Ebola, etc., or any other pathogens (e.g., airborne pathogens) which the pathogen detectors are configured to detect. In some embodiments, the locations at which the pathogens are detected are determined based on known locations of the pathogen detectors. For example, if a particular pathogen detector has a unique identification, the detection controller 502 may use the unique identification and a database to identify the location of the pathogen detector. In some embodiments, the detection results (e.g., data provided by the pathogen detectors) include information indicating the location of each pathogen detector. For example, each pathogen detector may report (e.g., to the detection controller 502) its location in the building (e.g., the building 10).

In some embodiments, the location of the pathogen detector changes (e.g., if the pathogen detector is mounted on a device, apparatus, or unit configured to translate throughout the building). The pathogen detector may be configured to wirelessly communicate to provide current pathogen detection data and current location in the building (e.g., wirelessly communicate with the detection controller 502). In some embodiments, the number of pathogen detection instances is a number of pathogen detections of any type of pathogen across all of the pathogen detectors in the building, a number of a particular type of pathogen across all of the pathogen detectors in the building, a number of pathogen detections of a single or multiple of the pathogen detectors over a time period (e.g., a 24 hour period), a number of pathogen detections of any or a particular type of pathogen of pathogen detectors in a particular area, etc.

In some embodiments, the magnitude of the pathogen detection is a value that is determined to quantify a severity of the pathogen detection. The magnitude can be determined based on any of, or a combination of, the type of pathogen detected, the location at which one or more pathogens are detected, the number of pathogen detection instances, etc. In some embodiments, the magnitude is determined by the detection controller 502.

Process 700 includes determining one or more responsive actions based on the type of pathogen detected, the location at which the pathogens are detected, the number of pathogen detection instances, and the magnitude of pathogen detection (step 706), according to some embodiments. In some embodiments, step 706 is performed by the detection controller 502 and/or one or more systems, devices, etc., that are communicably coupled with the detection controller 502 (e.g., the messaging system 508, the control system 510, the analytics system 512, the monitoring system 514, the service application system 516, the alert system 518, the HVAC system 100, etc.). In some embodiments, the responsive actions are determined by the detection controller 502 and provided to appropriate external systems that are configured to perform the responsive actions. In some embodiments, determining the responsive action includes determining a magnitude of the responsive action and determining a locale magnitude of the responsive action. The locale magnitude can be determined based on a location or configuration of the detector at which the pathogen is detected, according to some embodiments. In some embodiments, the magnitude of the responsive action is determined based on the type of pathogen detected at the detectors 504 and the number of instances of pathogen detection in the building 10.

The responsive actions can include any of, or any combination of, messaging actions, control actions, analytics actions, monitoring actions, service application initiations, alerting actions, adjustments to an HVAC system of the building, etc. The messaging actions can include any of providing a text message, an email, a notification, etc., to one or more occupants of the building, occupants of a particular zone of the building (e.g., where the pathogen is detected), employees that work in the building, etc. The control actions can include activation and/or determination of one or more infection control sequences (e.g., activating UV lights to kill pathogens in the building, increasing a fresh-air intake fraction of an AHU of the building, advanced filtration techniques, etc.). The control actions can be targeted to affect a particular zone or area of the building (e.g., based on the location of the detected pathogens, and/or a type of the detected pathogens). In some embodiments, a magnitude of the infection control sequences to be implemented is determined based on the magnitude of the pathogen detection (e.g., the detection of certain types of pathogens may require additional infection control sequences, etc.). The analytics actions can include using the detection results (e.g., real-world detection results) to update or adjust a predictive model (e.g., a Wells-Riley based predictive model) for use in determining high level control decisions to mitigate infection risks in the building. For example, the predictive model can include a deterministic portion and a stochastic adjustment, with the stochastic adjustment being updated or changed based on the detection results.

The monitoring actions can include generation of dashboards, user interfaces, reporting data, tabular data, graphs, graphical data, graphical user interfaces, etc., of the building. The monitoring actions can also include generation of an operation of any other system associated with the building that may be relevant to pathogenic presence in the building (e.g., what control sequences are implemented, potential infection reduction techniques, occupancy data in the building or different zones of the building, etc.). The dashboards, reporting data, tabular data, graphs, etc., can be presented to an administrator of the building.

The service application actions can include identifying, based on outputs of step 704 (or the detection controller 502), one or more service opportunities, according to some embodiments. In some embodiments, the service application initiations include scheduling and contracting of one or more services to address the service opportunities. Data can be collected from the implementation of the one or more services to generate baseline data, and subsequent data to identify if infection control sequences that are implemented in the building are effective.

The alerting actions can include determining that alarms or alerts should be provided to occupants of the building, according to some embodiments. The alarms or alerts can be targeted to specific areas, zones, rooms, floors, etc., of the building where a pathogen is detected. The types of alarms or alerts can be determined based on the type of pathogen detected and/or the magnitude of the pathogen detection.

Process 700 includes performing the one or more responsive actions using any of a messaging system, a control system, an analytics system, a monitoring system, a service application system, and alert system, or an HVAC system (step 708), according to some embodiments. In some embodiments, step 708 is performed by any of the messaging system 508, the control system 510, the analytics system 512, the monitoring system 514, the service application system 516, the alert system 518, or the HVAC system 100 of the building 10 (shown in FIG. 5).

The responsive actions may differ based on different applications of the process 700 or the pathogen detection system 500. For example, if the building of process 700 is an airport, and a particular type of pathogen is detected in the airport, the responsive actions may include sealing off the airport, alerting nearby aircrafts, shutting off outgoing transit, etc. In another example, if the building is a hospital, and a particular type of pathogen is detected in a certain area (e.g., a patient's room), the room may be sealed off, or caregivers may be prompted to use proper safety precaution (e.g., proper safety attire) before entering the room where the particular type of pathogen is detected. In another example, if the building is an office building, the responsive actions can include notifying employees of the office building (e.g., by providing a message) that a particular type of pathogen has been detected, and that all employees should work from home, or perform self-imposed quarantining. The responsive actions can further include restricting access to the office building (e.g., through adjustments to a badge security system of the building) until proper cleaning procedures have been performed and until an “all-clear” condition has been met (e.g., after waiting a predetermined amount of time). The responsive actions can also include, in such an example, providing a notification to the employees of the office building when the “all-clear” condition is met and when the employees can return to the office building.

It should be understood that while steps 706-708 describe multiple different types of responsive actions, process 700 does not require all of the responsive actions to be determined and performed. In some embodiments, steps 706-708 only include one or more of the responsive actions for the messaging system, the control system, the analytics system, the monitoring system, the service application system, the alert system, or the HVAC system. For example, the responsive actions may only include messaging actions, and consequently step 708 only includes “perform the responsive action using the messaging system.”

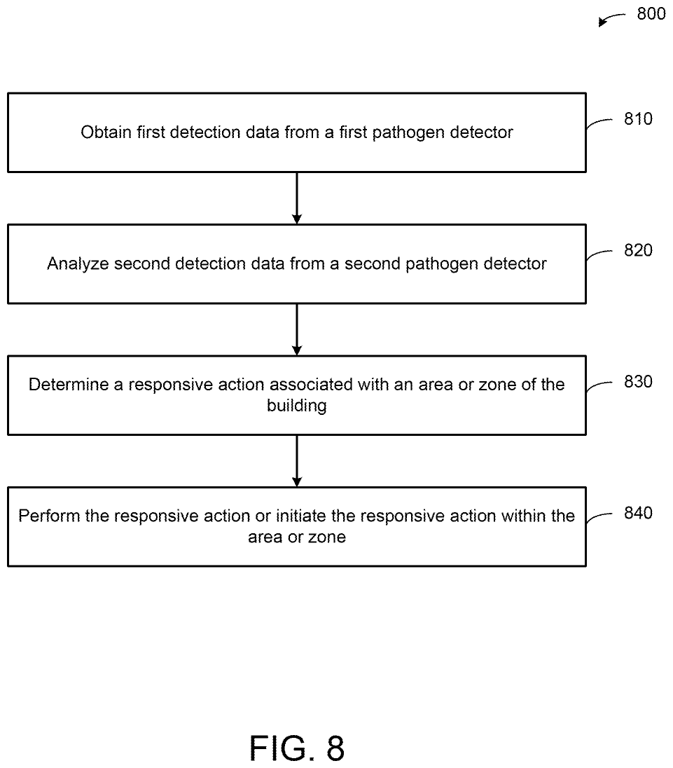

Referring to FIG. 8, a process 800 for performing biological sensing for a building is shown, according to some embodiments. The process 800 can be performed by the detection controller 502, and/or by the pathogen detection system 500 as described in greater detail above with reference to FIGS. 5-6. Process 800 includes steps 810-840, according to some embodiments. Process 800 can be performed using real-time or delayed detection data.

Process 800 includes obtaining first detection data from a first pathogen detector of a plurality of pathogen detectors positioned at a first location in a building (step 810), according to some embodiments. Additionally, process 800 also includes, in response to obtaining the first detection data, analyzing second detection data from a second pathogen detector of the plurality of pathogen detectors (step 820), according to some embodiments. The pathogen detectors can be pathogen detectors 504, according to some embodiments. In some embodiments, the pathogen detectors are or include static pathogen detectors (e.g., stationary and installed in a fixed location of the building). In some embodiments, the pathogen detectors are positioned at isolated rooms, entrances of the building, exits of the building, areas of the building with high expected occupancy density, etc. The pathogen detectors can also be positioned at areas of the building where pathogen detection is predicted to be high (e.g., in the bathroom, in a space of the building that is frequented by a large number of occupants, etc.). Locations of the pathogen detectors (e.g., floor, room, zone, etc.) may be known and can be used in steps 820-840, according to some embodiments. The pathogen detectors can also include a mobile unit including a pathogen detector that is configured to translate or move throughout the building, according to some embodiments. In some embodiments, step 810 is performed by the detection controller 502 and the pathogen detectors 504 as described in greater detail above with reference to FIGS. 5-6. Step 810 can also include obtaining seasonal infection or pathogen data for a region (e.g., a state, a country, a city, a country, a province, etc.) in which the building is located.

In some embodiments, process 800 may include obtaining data from multiple pathogen detectors placed at various heights within the building to account for the vertical distribution of airborne pathogens. This data can help in determining the concentration and distribution of pathogens in different sections of the building, such as higher or lower floors, and may be used to implement effective responsive actions. Additionally, process 800 may include obtaining data from pathogen detectors placed near air intake and exhaust points of the building's HVAC system. This can allow for the assessment of the efficiency of the HVAC system's filtration and pathogen reduction capabilities. In some embodiments, the pathogen detectors may be integrated with the HVAC system, allowing for real-time monitoring and adjustment of the system to enhance pathogen mitigation.

In some implementations, process 800 may include obtaining data from pathogen detectors placed in high-touch areas such as door handles, elevator buttons, and handrails. These detectors can be equipped with surface sampling capabilities to detect the presence of pathogens on frequently touched surfaces. This data can help in determining the need for increased sanitization measures or adjustments to cleaning schedules. In some embodiments, the pathogen detectors may be equipped with additional environmental sensors, such as temperature and humidity sensors. This data can be used to analyze the correlation between environmental conditions and pathogen presence, allowing for adjustments to be made to the building's environmental control systems to minimize the potential for pathogen survival and spread. Furthermore, process 800 may include obtaining data from pathogen detectors that are capable of detecting multiple types of pathogens, including bacteria, viruses, and fungi. The ability to detect various pathogens can provide a more comprehensive understanding of the building's overall pathogen profile and help in determining the most effective responsive actions. In some embodiments, the pathogen detectors may be combined with occupancy sensors, allowing for the detection of pathogens in relation to the number of occupants in the building or specific areas within the building. This information can be used to assess the risk of pathogen transmission based on occupancy levels and to implement responsive actions accordingly, such as restricting access to high-risk areas, adjusting HVAC settings, or increasing sanitization measures.

Referring to the pathogen detectors and pathogen detection system 500 generally, the plurality of pathogen detectors may include detectors with different capabilities and configurations to address various detection requirements. For instance, a first sensor located in a common area or multiple areas of the building may be configured to detect volatile organic compounds (VOCs) or other indicators of air quality, providing a broad assessment of the overall air quality within the building. In contrast, a second sensor located in a specific area or zone of the building may be designed to detect localized air quality issues, such as elevated carbon dioxide levels or high concentrations of airborne pathogens, within that particular area or zone. In particular, the pathogen detectors may include sensors for detecting pathogens in different mediums, such as air, surfaces, and water. For example, a first pathogen detector may be configured to detect the presence of pathogens in water supply lines, while a second pathogen detector may be designed to monitor the air quality in specific high-risk zones, such as restrooms or kitchens. Additionally, a third pathogen detector may be a surface-based sensor, capable of detecting pathogens on frequently touched surfaces, like door handles or countertops.

In some implementations, the pathogen detection system 500 may include mobile or portable pathogen detectors that can be easily deployed in different areas of the building based on changing requirements or concerns. These mobile detectors can be used to monitor air quality or detect pathogens on surfaces in real-time, providing valuable information on potential pathogen hotspots within the building. Examples of mobile detectors include handheld devices, robotic systems, or wearable sensors that can be worn by occupants as they move around the building. Furthermore, the pathogen detection system 500 may be integrated with other building systems, such as security systems or access control systems, to monitor and control the movement of occupants within the building based on pathogen detection data. For instance, access to certain areas of the building may be restricted or limited based on the detection of pathogens in those areas, ensuring the safety and well-being of the occupants. In some embodiments, the pathogen detectors may be equipped with machine learning algorithms or artificial intelligence capabilities to continuously analyze the collected data and improve the accuracy and efficiency of pathogen detection. This can enable the system to better predict potential pathogen outbreaks, identify trends, and optimize the deployment of responsive actions within the building.

In various embodiments, the pathogen detection system 500 can request additional detection data from the first pathogen detector, the second pathogen detector, or the third pathogen detector based on receiving a positive detection from at least one of the plurality of pathogen detectors. Additionally, the pathogen detection system 500 can prioritize a responsive action based on the difference in pathogen detection between the detected information impacted by the overall air quality or the overall sewage conditions within the building and the detected information impacted by the localized air quality or the localized sewage conditions within the area or zone.

In various embodiments, the pathogen detection system 500 can dynamically adjust its monitoring and response strategies based on the detection data collected from the different pathogen detectors. For instance, upon receiving a positive detection from at least one of the plurality of pathogen detectors, the system can request additional detection data from the other detectors to gain a more comprehensive understanding of the pathogen presence in the building. This additional data may be used to refine the system's assessment of the risk level and inform subsequent responsive actions. In some cases, the pathogen detection system 500 can prioritize responsive actions based on the differences in pathogen detection results between detectors monitoring the overall air quality or sewage conditions within the building and those focused on localized conditions within specific areas or zones. For example, if a significant discrepancy is observed between the overall and localized pathogen detection results, the system may prioritize actions targeting the affected area or zone to mitigate the risk of further contamination.

Responsive actions may include, but are not limited to, adjusting ventilation systems to increase air exchange, activating air purification systems, implementing more frequent cleaning and sanitization protocols, isolating affected areas, or temporarily restricting access to certain spaces within the building. In some embodiments, the pathogen detection system 500 may be configured to automatically initiate these responsive actions, while in other cases, the system may generate alerts or recommendations for building management to manually implement the suggested actions. To further enhance the effectiveness of the pathogen detection system, alternative or supplementary detection methods may be employed in conjunction with the primary pathogen detectors. Examples of such methods include deploying additional mobile or portable detectors, integrating data from occupants' personal wearable devices, UV-C lighting, or utilizing crowd-sourced information from social media or other online sources to identify potential pathogen outbreaks. In some embodiments, the pathogen detection system 500 may incorporate machine learning algorithms or artificial intelligence techniques to analyze the collected data, identify patterns or trends, and predict potential pathogen outbreaks or contamination risks. By continuously updating and refining its predictive models based on the real-time detection data, the system can proactively implement responsive actions, optimizing the overall effectiveness of the pathogen detection and mitigation process within the building.

In some embodiments, he second pathogen detector can be configured to monitor a different area, zone, or aspect of the building compared to the first pathogen detector (step 810). In some embodiments, the second pathogen detector can be configured to focus on specific locations or zones within the building, such as isolated rooms, high occupancy areas, or areas with a higher likelihood of pathogen transmission (e.g., restrooms, cafeterias, or shared workspaces). By analyzing the second detection data in conjunction with the first detection data, the pathogen detection system 500 can gain a better understanding of the overall pathogen situation within the building and identify localized areas of concern that may require targeted responsive actions. In some cases, the second pathogen detector may be configured to monitor a different type of environmental parameter or pathogen transmission medium compared to the first pathogen detector. For example, while the first pathogen detector may be focused on detecting pathogens within the air, the second pathogen detector may be designed to monitor the presence of pathogens within sewage or on surfaces. By analyzing data from detectors with different monitoring modalities, the pathogen detection system 500 can obtain a more holistic view of the pathogen risk within the building and implement more effective mitigation strategies. In various embodiments, the analysis of the second detection data can involve comparing the data against predetermined thresholds or benchmarks to determine the risk level associated with the pathogen presence.

Process 800 includes determining a responsive action associated with an area or zone of the building based on the first detection data and the second detection data (Step 830), according to some embodiments. Additionally, process 800 includes performing the responsive action or initiate the responsive action within the area or zone (step 840), according to some embodiments. In some embodiments, the responsive action can include implementing measures to mitigate pathogen spread, such as increasing air circulation or adjusting humidity levels in certain areas or zones of the building. Alternatively, or in combination, the responsive action may involve temporarily restricting access to specific areas where elevated pathogen levels have been detected, or implementing more frequent cleaning and sanitization protocols in those locations. Additionally, process 800 includes performing the responsive action or initiating the responsive action within the area or zone (step 840), according to some embodiments. In some cases, the pathogen detection system 500 can be integrated with other building systems, such as the building automation system (BAS), to automatically initiate the responsive action. This can help ensure a timely response to potential pathogen risks and minimize the impact on occupants and operations within the building.

In some embodiments, the responsive action includes a control action, and the processing circuitry of the pathogen detection system can include a control system configured to initiate one or more infection control sequences through operation of an infection control system of the building to perform the control action. For example, the one or more infection control sequences can include at least one of an adjustment to a fresh air intake of an air handling unit (AHU) of a heating, ventilation, or air conditioning (HVAC) system of the building, activation of one or more ultraviolet (UV) lights to disinfect return air from a zone of the building, or initiating one or more filtration techniques to filter air in the building. In various embodiments, the responsive action may also involve notifying building occupants or management about the detected pathogen risk and providing guidance on appropriate precautions or actions to take, such as wearing masks, practicing social distancing, or seeking medical attention if symptoms develop. This can help empower individuals to take proactive measures to protect themselves and others, while also fostering a sense of transparency and trust between the building management and occupants.

In some embodiments, the pathogen detection system can monitor additional detection data from the first pathogen detector and the second pathogen detector, wherein monitoring the additional detection data includes adjusting a frequency of sampling and analysis based on at least one of previous analyses, previously collected detection data, building occupancy, or pathogen community data. By adapting the sampling frequency and analysis to various factors, the pathogen detection system can optimize its efficiency, effectiveness, and accuracy in identifying potential pathogen risks. For example, if a significant increase in building occupancy is expected, such as during a large event or conference, the pathogen detection system may adjust the sampling frequency to more closely monitor the potential for pathogen spread. Similarly, if historical data or pathogen community data suggest a higher risk of specific pathogens during certain times of the year, the system can adjust its focus to monitor for those pathogens more closely during those periods.

In some embodiments, the pathogen detection system may request additional detection data from the mobile sensor exclusively based on receiving the second detection data from the second pathogen detector, such as an airborne sensor, without considering the data from the first pathogen detector, like a sewage sensor. For example, if the second pathogen detector detects an elevated level of airborne pathogens in a specific area of the building, the detection controller may then request additional detection data from the mobile sensor to further investigate the presence, concentration, or spread of the pathogen in the air within the building. In this scenario, the mobile sensor can be deployed to collect and analyze samples from various locations in the building, such as high-traffic areas, ventilation systems, or other zones where the airborne pathogen levels may be elevated. By focusing on the additional detection data from the mobile sensor and the second detection data from the airborne sensor, the pathogen detection system can effectively determine a pattern or trend associated with the presence, concentration, or spread of the airborne pathogen within the building. This targeted approach enables a more efficient allocation of resources and timely implementation of responsive actions to limit the spread of the pathogen and protect the building's occupants.

In some embodiments, the pathogen detection system is configured to determine a severity or magnitude of the pathogen in the building and/or a locality of areas of the pathogen in the building using the pathogen data. In various embodiments, the pathogen detection system can provide visualizations or reports of the pathogen data, including the severity, magnitude, and locality of the detected pathogens. These visualizations or reports can be shared with building management or occupants, enabling them to better understand the pathogen risks present in the building and to make informed decisions about their activities and behaviors. In certain embodiments, the pathogen detection system can also communicate (e.g., via wired or wireless communication) with external databases or systems to compare the detected pathogen data with regional or global trends. This can help put the detected risks into context and facilitate more informed decision-making and response planning at the building level. Additionally, the system can contribute its own pathogen data to these external sources, thereby improving the overall understanding of pathogen spread and dynamics in the broader community.

In some embodiments, a pathogen detection system 500 for a building can include a first pathogen detector configured to obtain a first sample, the first pathogen detector positioned at a first location in the building, a second pathogen detector configured to obtain a second sample, wherein the first pathogen detector and the second pathogen detector output pathogen data indicating whether presence of a pathogen has been detected, and a detection controller configured to assess the pathogen data and determine a responsive action associated with an area or zone of the building based on the first sample and the second sample.

In some embodiments, the first pathogen detector is a sewage sampling system configured to take a sample of sewage from a sewage line or a sewage outlet of the building and sense the presence of the pathogen within the sample of sewage. In some embodiments, the second pathogen detector is an air sensor configured to take a sample of air from one or more areas or zones of the building and sense the presence of the pathogen within the sample of air.

In some embodiments, the first pathogen detector, which can be a sewage sampling system, can be designed with various sampling methods to collect sewage samples effectively. These methods may include passive sampling, where sewage is continuously monitored as it flows through the sewage line or outlet, or active sampling, where a discrete sample is collected at predetermined intervals or in response to specific triggers. The sewage sampling system can incorporate advanced technologies such as microfluidics, lab-on-a-chip, or automated sampling devices to enhance the accuracy and efficiency of pathogen detection within the collected sewage samples. The sewage sampling system may also include sensors capable of detecting specific pathogens or groups of pathogens, such as bacteria, viruses, fungi, or parasites. These sensors can utilize a variety of detection techniques, such as nucleic acid-based detection (e.g., PCR, isothermal amplification), immunoassays (e.g., ELISA, lateral flow), or biosensors (e.g., electrochemical, optical, or acoustic sensors). The sewage sampling system can further be equipped with communication capabilities, allowing it to transmit the pathogen detection data to the detection controller for analysis and decision-making.

In some embodiments, the second pathogen detector, which can be an air sensor, can be designed to collect air samples from one or more areas or zones within the building. The air sensor may utilize various air sampling techniques, such as active air sampling with a high-volume air sampler or passive air sampling with settling plates or impaction devices. The air sensor can also be equipped with sensors capable of detecting specific pathogens or groups of pathogens, similar to the sewage sampling system. The air sensor may employ various detection techniques, such as nucleic acid-based detection, immunoassays, or biosensors, to identify the presence of pathogens in the collected air samples. These techniques may be designed to operate in real-time, providing continuous monitoring of air quality within the building, or they may be used to analyze discrete samples collected at predetermined intervals or in response to specific triggers. The air sensor can also be equipped with communication capabilities, enabling it to transmit the pathogen detection data to the detection controller for analysis and decision-making. The combination of the first pathogen detector (sewage sampling system) and the second pathogen detector (air sensor) allows for a comprehensive assessment of pathogen presence within the building, ensuring a more accurate and effective response to potential threats.

In an example, a sewage sampling system can be integrated into a building to monitor both localized conditions in the bathrooms and overall sewage conditions in the main output pipe of the building. To achieve this, two types of sewage sampling systems can be installed: one for localized sampling within individual bathrooms and another for obtaining overall sewage conditions in the main output pipe.

For localized conditions, sewage sampling sensors can be installed within the drainage pipes of each bathroom in the building. These sensors can be strategically placed to monitor sewage outflow from specific fixtures, such as toilets, urinals, and sinks, allowing for a more precise analysis of pathogen presence in these localized areas. The sewage sampling system within the bathrooms can collect samples at predetermined intervals or upon detecting a sewage flow event. The collected samples are then analyzed for the presence of pathogens, with the results transmitted to the detection controller.

For overall sewage conditions, a sewage sampling system can be integrated into the main output pipe of the building. This system can consist of a sampling device that extracts representative sewage samples from the main output pipe, capturing a mixture of sewage from all the bathrooms and other wastewater sources within the building. The sampling device can be configured to collect samples at regular intervals or in response to a specific trigger, such as an increase in pathogen levels detected by the localized sensors.

In the above example, the detection controller can then analyze the data obtained from both the localized and overall sewage sampling systems. By comparing the pathogen levels in the individual bathrooms to those in the main output pipe, the detection controller can identify potential hotspots within the building and implement responsive actions accordingly. These actions may include increasing the frequency of cleaning and disinfection in affected bathrooms, adjusting sewage treatment processes, or notifying building occupants and management of potential risks. This comprehensive approach, combining localized and overall sewage monitoring, enables the pathogen detection system to provide a more accurate and effective response to potential pathogen threats within the building.

In addition to monitoring localized and overall sewage conditions (or any other conditions) within the building, the pathogen detection system can also incorporate sewage data from the public sewer system or wastewater treatment facilities to provide a broader perspective on pathogen levels in the surrounding community. Public health agencies or wastewater treatment operators may collect and analyze sewage samples from various locations in the community to monitor pathogen presence and identify trends in infection rates. By comparing the data obtained from the building's localized and overall sewage conditions to the public sewage data, the detection controller can better understand the building's pathogen situation within the context of the larger community. For example, if elevated pathogen levels are detected in both the building's sewage and the public sewer system, it may indicate a widespread issue that requires coordinated efforts from public health authorities and building management to address. On the other hand, if elevated pathogen levels are found only within the building, the detection controller can focus on implementing responsive actions specific to the building's needs.

It is important to understand that the example provided above, focused on comparing sewage conditions, is just one instance of how the pathogen detection system can be integrated to collect various types of data. In practice, the system can be designed to collect localized and overall data related to sewage conditions, air quality, surface contamination, or any combination thereof, as well as public data from external sources. By gathering and analyzing a wide range of data, the pathogen detection system can provide a more comprehensive understanding of the pathogen situation within the building and its surrounding community. This versatile approach allows the detection controller to determine responsive actions based on a holistic view of the pathogen presence and potential risks. By considering localized, overall, and public data across multiple dimensions such as sewage, air, and surface, the pathogen detection system can more accurately identify areas of concern and implement targeted, effective measures to address them. Furthermore, the integration of diverse data sources enables better collaboration between building management, public health authorities, and other stakeholders, promoting a coordinated and efficient response to minimize the spread of pathogens and protect the health of building occupants and the larger community.

In various embodiments, the pathogen detection system may incorporate additional pathogen detectors or types of pathogen detectors to provide coverage and monitoring within the building. For example, the system could include surface swab sensors to detect pathogens on commonly touched surfaces, or deploy portable or wearable sensors for individual occupants to monitor their immediate environment. In certain embodiments, the detection controller can be configured to analyze and compare the pathogen data from the first and second pathogen detectors, as well as any additional detectors in the system, in order to determine the most appropriate responsive action. This can include, for example, identifying specific areas or zones of the building with higher pathogen concentrations, implementing targeted cleaning or disinfection measures, adjusting HVAC settings to improve air quality, or informing occupants of potential risks and recommended precautions.

In some embodiments, the pathogen detection system further includes a mobile sensor configured to sense the presence of the pathogen on a surface or within air. This mobile sensor can be a handheld device, a robot, or even an integrated wearable sensor that allows for on-the-go monitoring of pathogen presence in various parts of the building. In some embodiments, the detection controller is further configured to request additional detection data from the first pathogen detector, the second pathogen detector, or the mobile sensor based on receiving a positive detection from at least one of the first pathogen detector, the second pathogen detector, or the mobile sensor. In some embodiments, the first pathogen detector is located in a common area or multiple areas of the building and configured to detect information impacted by overall air quality or overall sewage conditions within the building, while the second pathogen detector is located in the area or zone of the building and configured to detect information impacted by localized air quality or localized sewage conditions within the area or zone. In some embodiments, the detection controller is further configured to prioritize the responsive action based on the difference in pathogen detection between the detected information impacted by the overall air quality or the overall sewage conditions within the building and the detected information impacted by the localized air quality or the localized sewage conditions within the area or zone.

In some embodiments, the pathogen detection system may be integrated with other building systems or external systems to enhance the efficiency and effectiveness of the response to the detected presence of pathogens. The transceiver within the pathogen detection system can transmit messages to these external systems, providing real-time updates on the pathogen detection status in the building. Examples of external systems that can be integrated with the pathogen detection system include building automation systems, security systems, access control systems, or public health monitoring systems. These external systems may take additional actions to mitigate the spread of the detected pathogens, such as adjusting the operation of the HVAC system to improve air quality, modifying access controls to limit the movement of occupants within the building, or notifying public health authorities to initiate a broader response.

In some embodiments, the detection controller may generate messages that convey information about the presence or absence of the pathogen in the first sample or the second sample. These messages can also include details about the specific locations of the first and second pathogen detectors within the building, allowing for targeted responses to areas where the pathogen is detected. The detection controller may use this information to update the locations of the pathogen detectors, repositioning them to areas with higher pathogen risk or where additional monitoring is needed. In some embodiments, the detection controller can coordinate the responsive actions within the building, ensuring that the appropriate measures are taken to minimize the spread of pathogens. These responsive actions may include adjusting ventilation systems, activating air purification technologies, initiating cleaning or disinfection protocols, or restricting access to affected areas.

### Example Applications

Referring generally to the FIGURES, the systems and methods described herein can be applied in a variety of applications or for different types of buildings. For example, the building 10 may be an airport, an incarceration site, a cruise ship, a hotel, a nursing home, or an assisted living facility, among others. Depending on the type of the building 10 and the application thereof, the responsive actions initiated by the detection controller 502 may differ. For instance, if the building 10 is an airport, one of the responsive actions may be to shut down travel or delay flights in response to detecting a particular type of pathogen in the airport. The pathogen detection system can incorporate detection data from various pathogen detectors, including those monitoring localized and overall air quality, surface contamination, and sewage conditions, as well as public data from external sources. By analyzing this dataset, the detection controller 502 can determine the most appropriate responsive action to minimize the spread of the pathogen.

In the case of higher-risk facilities for infection spread (e.g., nursing homes, assisted living homes, etc.), the responsive actions can include shutting down or limiting regular facility operations to limit or reduce infective spread. These responsive actions may be determined by evaluating the detection data from multiple pathogen detectors placed throughout the facility, along with public health data, to identify areas of concern and target specific locations for intervention. As the pathogen detection system is adaptable to various building types and applications, it can contribute to a safer and healthier environment for occupants and visitors, while promoting a coordinated and efficient response to minimize the impact of pathogens on the larger community.

### Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied and the nature or number of discrete elements or positions may be altered or varied. Accordingly, all such modifications are intended to be included within the scope of the present disclosure. The order or sequence of any process or method steps may be varied or re-sequenced according to alternative embodiments. Other substitutions, modifications, changes, and omissions may be made in the design, operating conditions and arrangement of the exemplary embodiments without departing from the scope of the present disclosure.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can include RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

What is claimed is:

1. A pathogen detection system for a building, comprising:
a plurality of pathogen detectors positioned in the building at a plurality of locations, the plurality of pathogen detectors configured to output pathogen data indicating whether presence of a pathogen has been detected; and
processing circuitry configured to:
obtain first detection data from a first pathogen detector of the plurality of pathogen detectors positioned at a first location in the building;
in response to obtaining the first detection data, analyze second detection data from a second pathogen detector of the plurality of pathogen detectors;
determine a responsive action associated with an area or zone of the building based on the first detection data and the second detection data; and
perform the responsive action or initiate the responsive action within the area or zone.

2. The pathogen detection system of claim 1, wherein the first pathogen detector is configured to sense the presence of the pathogen within sewage, and the second pathogen detector is configured to sense the presence of the pathogen within air.

3. The pathogen detection system of claim 2, wherein the first pathogen detector is positioned within a sewage line or sewage output of the building, and wherein the second pathogen detector is positioned on a surface of the building.

4. The pathogen detection system of claim 2, wherein a third pathogen detector is a mobile sensor configured to sense the presence of the pathogen on a surface or within air, and wherein the processing circuitry is further configured to:
request additional detection data from the first pathogen detector, the second pathogen detector, or the third pathogen detector based on receiving a positive detection from at least one of the plurality of pathogen detectors.

5. The pathogen detection system of claim 3, further comprising:
request additional detection data from the mobile sensor based on receiving the second detection data from the second pathogen detector; and
determine a pattern or trend in both the additional detection data and the second detection data, wherein the pattern or trend is associated with at least one of sensing the presence of the pathogen, a concentration of the pathogen, or a spread of the pathogen within the building.

6. The pathogen detection system of claim 1, wherein the plurality of pathogen detectors comprises a first sensor located in a common area or multiple areas of the building configured to detect information impacted by overall air quality or overall sewage conditions within the building, and wherein a second sensor located in the area or zone of the building configured to detect information impacted by localized air quality or localized sewage conditions within the area or zone.

7. The pathogen detection system of claim 6, wherein the processing circuitry is further configured to prioritize the responsive action based on the difference in pathogen detection between the detected information impacted by the overall air quality or the overall sewage conditions within the building and the detected information impacted by the localized air quality or the localized sewage conditions within the area or zone.

8. The pathogen detection system of claim 1, the processing circuitry is further configured to:
monitor additional detection data from the first pathogen detector and the second pathogen detector, wherein monitoring the additional detection data comprises adjusting a frequency of sampling and analysis based on at least one of previous analyses, previously collected detection data, building occupancy, or pathogen community data; and
wherein the pathogen detection system is configured to determine a severity or magnitude of the pathogen in the building and/or a locality of areas of the pathogen in the building using the pathogen data.

9. The pathogen detection system of claim 1, wherein the responsive action comprises a control action and the processing circuitry comprises a control system configured to initiate one or more infection control sequences through operation of an infection control system of the building to perform the control action.

10. The pathogen detection system of claim 9, wherein the one or more infection control sequences comprising at least one of an adjustment to a fresh air intake of an air handling unit (AHU) of a heating, ventilation, or air conditioning (HVAC) system of the building, activation of one or more ultraviolet (UV) lights to disinfect return air from a zone of the building, or initiating one or more filtration techniques to filter air in the building.

11. A pathogen detection system for a building, comprising:
a first pathogen detector configured to obtain a first sample, the first pathogen detector positioned at a first location in the building;
a second pathogen detector configured to obtain a second sample, wherein the first pathogen detector and the second pathogen detector output pathogen data indicating whether presence of a pathogen has been detected; and
a detection controller configured to assess the pathogen data and determine a responsive action associated with an area or zone of the building based on the first sample and the second sample.

12. The pathogen detection system of claim 11, wherein the first pathogen detector is a sewage sampling system configured to take a sample of sewage from a sewage line or a sewage outlet of the building and sense the presence of the pathogen within the sample of sewage.

13. The pathogen detection system of claim 12, wherein the second pathogen detector is an air sensor configured to take a sample of air from one or more areas or zones of the building and sense and sense the presence of the pathogen within the sample of air.

14. The pathogen detection system of claim 13, further comprising:
a mobile sensor configured to sense the presence of the pathogen on a surface or within air; and
wherein the detection controller is further configured to request additional detection data from the first pathogen detector, the second pathogen detector, or the mobile sensor based on receiving a positive detection from at least one of the first pathogen detector, the second pathogen detector, or the mobile sensor.

15. The pathogen detection system of claim 11, wherein the first pathogen detector is located in a common area or multiple areas of the building and configured to detect information impacted by overall air quality or overall sewage conditions within the building, and the second pathogen detector is located in the area or zone of the building and configured to detect information impacted by localized air quality or localized sewage conditions within the area or zone.

16. The pathogen detection system of claim 15, wherein the detection controller is further configured to prioritize the responsive action based on the difference in pathogen detection between the detected information impacted by the overall air quality or the overall sewage conditions within the building and the detected information impacted by the localized air quality or the localized sewage conditions within the area or zone.

17. The pathogen detection system of claim 11, further comprising:
a transceiver configured to transmit a message to an external system; and
the external system configured to take one or more additional actions to detect or limit a spread of the pathogen in the building responsive to receiving the message from the transceiver.

18. The pathogen detection system of claim 11, wherein the detection controller is further configured to generate a message indicating at least one of a presence or absence of the pathogen in the first sample or the second sample, and update a plurality of locations of the first pathogen detector or the second pathogen detector within the building based on the presence of the pathogen in the first sample or the second sample.

19. The pathogen detection system of claim 11, wherein the detection controller is further configured to perform the responsive action or initiate the responsive action within the area or zone.

20. A method comprising:
obtaining, by a pathogen detection system, first detection data from a first pathogen detector of a plurality of pathogen detectors positioned at a first location in a building;
in response to obtaining the first detection data, analyzing, by the pathogen detection system, second detection data from a second pathogen detector of the plurality of pathogen detectors;
determining, by the pathogen detection system, a responsive action associated with an area or zone of the building based on the first detection data and the second detection data; and
performing, by the pathogen detection system, the responsive action or initiate the responsive action within the area or zone.
